(19) United States
(12) Patent Application Publication
(10) Pub. No.: US 2004/0143101 A1
(43) Pub. Date: Jul. 22, 2004

(54) Immunoglobulin construct containing anti-mucin variable domain sequences for eliciting an anti-idiotype anti-tumor response

(76) Inventors: Daniel A. Soltis, Belle Mead, NJ (US); Ronald M Burch, Wilton, CT (US); Rajiv Shukla, Metuchen, NJ (US)

Correspondence Address: DARBY & DARBY P.C., P. O. BOX 5257, NEW YORK, NY, 10150-5257, US

(21) Appl. No.: 10/469,125

(22) PCT Filed: Apr. 2, 2002
(86) PCT No.: PCT/US02/10304

(30) Foreign Application Priority Data
Apr. 2, 2001 (US) 60281182

(51) Int. Cl. C07K 16/30

(52) U.S. Cl. 530/388.22

(57) ABSTRACT

The present invention provides a variant of an immunoglobulin variable domain including (A) at least one CDR region and (B) framework regions flanking the CDR region, wherein the variant also contains (a) a CDR region having added or substituted therein at least on binding sequence and (b) the flanking framework regions, wherein the binding sequence is heterelogous to the CDR and is an antigenic sequence from a MUC-1 binding sequence.

### FIELD OF THE INVENTION

The present invention relates to products and methods to treat cancer to elicit an anti-idiotype response targeted to tumor cells that bear mucin-like glycoproteins present on neoplasms of epithelial origin including carcinomas of the breast, ovary and gastrointestinal tract. In particular, a vaccine based on the sequence of the HMFG-1 monoclonal antibody may prove beneficial in both the treatment and possible prevention of breast cancer and other epithelial-derived tumors that express the antigen recognized by HMFG-1.

### BACKGROUND OF THE INVENTION

Cancer remains the second leading cause of death in the United States. There were an estimated 563,100 cancer deaths in 1999. Each year, about 1,222,000 new cancer cases are diagnosed

Non-surgical therapy for breast, lung, colon, and ovarian cancers, as well as many other solid tumors, is presently poor. While initial therapies for breast and ovarian cancer with taxanes result in some response by most patients, nearly all patients with ovarian cancer and some patients with breast cancer relapse.

There is substantial evidence indicating that the immune system plays a critical role in the prevention of cancer and the control of tumor growth. This includes the occasional observation of spontaneous tumor regression, the correlation of spontaneous regressions with the presence of tumor-infiltrating lymphocytes (TILs) and the identification of TILs that are specific for tumor antigens. However, as evidenced by the incidence rates of cancer, the immune response is often not sufficient to successfully combat the tumor. During the past 40 years, an increased understanding of the immune mechanism has led to the development of approaches to enhance the immune response against the tumor. These have included the expression in tumor cells of genes encoding immunostimulatory molecules and several approaches to enhance tumor antigen presentation. Unfortunately, only limited indications of beneficial effects have been seen and attempts to utilize these new approaches to immunotherapy have been disappointing.

In recent years, there has been a renewed interest in the development of cancer vaccines. This has resulted in part from the identification of new tumor-specific antigens and an increased understanding of the importance of antigen presentation and lymphocyte activation. Here we propose to produce an antibody vaccine using a mouse mAb, HMFG-1, that has been shown to react with a number of carcinomas of epithelial origin and has been used previously for tumor imaging and antibody-guided irradiation of tumors.

### Mucins

The human milk fat globule (HMFG) membrane contains several glycoproteins, termed mucins, that are frequently expressed at higher levels in breast and other adenocarcinomas than in normal tissue. One of these mucins, referred to as MUC-1, has been found to be overexpressed in tumor cells particularly in an aberrantly glycosylated form. Numerous monoclonal antibodies (mAbs) have been prepared against these cell-surface antigens that have proven to be useful in the diagnosis and treatment of breast cancer including two mAbs termed HMFG-1 and HMFG-2. In particular, HMFG-1, has been shown to recognize determinants on mucin-like glycoproteins present on neoplasms of epithelial origin including carcinomas of the breast, ovary and gastrointestinal tract. A vaccine based on the sequence of the HMFG-1 monoclonal antibody may prove beneficial in both the treatment and possible prevention of breast cancer and other epithelial-derived tumors that express the antigen recognized by HMFG-1.

### Immunoglobulins and Anti-Idiotype Immune Response

The basic unit of antibody immunoglobulin structure is a complex of four polypeptides—two identical low molecular weight or “light” chains and two identical high molecular weight or “heavy” chains—linked together by both non-covalent associations and by disulfide bonds. Each light and heavy chain of an antibody has a variable region at its amino terminus and a constant domain at its carboxyl terminus. The variable regions are distinct for each antibody and contain the antigen binding site. Each variable domain is comprised of four relatively conserved framework regions and three regions of sequence hypervariability termed complementarity determining regions or “CDRs”. For the most part, it is the CDRs that form the antigen binding site and confer antigen specificity. The constant domains are more highly conserved than the variable regions, with slight variations due to haplotypic differences.

Based on their amino acid sequences, light chains are classified as either kappa or lambda. The constant region of heavy chains is composed of multiple domains (CH1, CH2, CH3 . . . CHx), the number depending upon the particular antibody class. The CH1 region is separated from the CH2 region by a hinge region that allows flexibility in the antibody. The variable region of each light chain aligns with the variable region of each heavy chain, and the constant region of each light chain aligns with the first constant region of each heavy chain. The CH2-CHx domains of the constant region of a heavy chain form an “Fc region” that is responsible for the effector functions of the immunoglobulin molecule, such as complement binding and binding to the Fc receptors expressed by lymphocytes, granulocytes, monocyte lineage cells, killer cells, mast cells, and other immune effector cells.

In modern medicine, immunotherapy or vaccination has virtually eradicated diseases such as polio, tetanus, tuberculosis, chicken pox, measles, hepatitis, etc. The approach using vaccinations has exploited the ability of the immune system to prevent infectious diseases.

There are two types of immunotherapy, active immunotherapy and passive immunotherapy. In active immunotherapy, an antigen is administered in a vaccine to a patient so as to elicit a long-lasting protective immune response against the antigen. Passive immunotherapy involves the administration of protective antibodies to a patient to elicit an acute immune response that lasts only as long as the antibody is present. Antibody therapy is conventionally characterized as passive since the patient is not the source of the antibodies. However, the term passive may be misleading because a patient may produce anti-idiotype secondary antibodies, which in turn provoke an immune response that is cross-reactive with the original antigen. Immunotherapy where the patient generates anti-idiotypic antibodies is often more therapeutically effective than passive immunotherapy because the patient's own immune system generates an active immune response against cells bearing the particular antigen well after the initial infusion of protective antibody has cleared from the system.

In an anti-idiotypic response, antibodies produced initially during an immune response or introduced into an organism will carry unique new epitopes to which the organism is not tolerant, and therefore will elicit production of secondary antibodies (termed “Ab2”), some of which are directed against the idiotype (i.e., the antigen binding site) of the primary antibody (termed “Ab1”), i.e., the antibody that was initially produced or introduced exogenously. These secondary antibodies or Ab2, likewise will have an idiotype that will induce production of tertiary antibodies (termed “Ab3”), some of which will recognize the antigen binding site of Ab2, and so forth. This is known as the “network” theory. Some of the secondary antibodies will have a binding site that is an analog of the original antigen, and thus will reproduce the “internal image” of the original antigen. Tertiary or Ab3 antibodies that recognize this antigen binding site of the Ab2 antibody will also recognize the original antigen.

Therefore, anti-idiotypic antibodies have binding sites that are similar in conformation and charge to the antigen, and can elicit the same or a greater response than that of the target antigen itself. Administration of an exogenous antibody that can elicit a strong anti-idiotypic response can thus serve as an effective vaccine, by eliciting a self-propagating anti-idiotype immune response.

Clinically useful anti-idiotypic responses are rare when intact antibodies are used as the immunogen. The ability to deliver antibodies that reproducibly cause the generation of such an anti-idiotypic response is difficult (Foon, et al., J. Clin. Invest. 1995, 9:334-342; Madiyalakan, et al., Hybridoma 1995, 14: 199-203). One of the reasons for the failure generally to generate an anti-idiotypic response is that Ab1, while exogenous, is still very similar to “self”, as all antibodies have very similar structure and anti-idiotypic responses to self molecules tend to be very limited. A strong anti-idiotypic cascade has been observed when Ab1 has been structurally damaged (Madiyalakan et al., Hybridoma, 1995, 14:199-203) rendering the antibody more foreign. U.S. Pat. No. 4,918,164 discloses direct administration to the subject of exogenously produced anti-idiotype antibodies that are raised against the idiotype of an anti-tumor antibody. After administration, the subject produces anti-antibodies that not only recognize these anti-idiotype antibodies, but also recognize the original tumor epitope, thereby directing complement activation and other immune system responses to a foreign entity to attack the tumor cell that expresses the tumor epitope.

PCT Publication WO 99/25378 relates to synthebody molecules, particularly antibodies, that bind one member of a binding pair and have at least one complementarity determining region (CDR) that contains the amino acid sequence of a binding site for that member of the binding pair. The binding site is derived from the other member of the binding pair. It also relates to methods for treating, diagnosing, or screening for diseases and disorders associated with the expression of the member of the binding pair using the modified antibodies.

PCT Publication WO 99/25379 relates to vaccine compositions of antibodies in which one or more variable region cysteine residues that form intrachain disulfide bonds have been replaced with amino acid residues that do not contain a sulfhydryl group and, therefore, do not form disulfide bonds. It also relates to use of the vaccine compositions to treat or prevent certain diseases and disorders.

In sum, there is a need in the art to identify effective methods to target mucin, particularly MUC-1, bearing tumor cells. In particular, there is a need to generate an effective anti-tumor immune response. The present invention addresses these and other needs in the art with the discovery of an effective anti-idiotype antibody-based mucin-specific vaccine.

### OBJECTS OF THE INVENTION

It is therefore an object of the present invention to provide variants of an immunoglobulin variable domain (constructs). The immunoglobulin variable domain includes (A) at least one CDR region and (B) framework regions flanking said CDR. The variant includes (a) the CDR region having added or substituted therein at least one binding sequence and (b) the flanking framework regions, preferably including a disrupted intrachain disulfide bond, wherein the binding sequence is heterologous to the CDR and is a binding sequence from a binding site of a binding pair, and wherein said binding sequence is an MUC-1-binding portion of an anti-mucin monoclonal antibody. In a preferred embodiment, components (a) and (b) of the variant are contained within a sequence encoding the heavy and light chain variable region, preferably including a disrupted intrachain disulfide bond.

In further embodiment, the present invention provides a construct having (i) one or more amino acid residues in one or more of the flanking framework regions has been substituted or deleted, (ii) one or more amino acid residues has been added in one or more of the flanking framework regions, or (iii) a combination of (i) and (ii). Alternatively, the construct has (i) one or more amino acid residues in one or more framework regions other than the framework regions flanking the CDR has been substituted or deleted, (ii) one or more amino acid residues has been added in one or more framework regions other than the framework regions flanking said CDR, or (iii) a combination of (i) and (ii). In yet another alternative, the construct has (i) one or more amino acid residues in one or more of the flanking framework regions has been substituted or deleted, (ii) one or more amino acid residues has been added in one or more of the flanking framework regions, or (iii) a combination of (i) and (ii); and (iv) one or more amino acid residues in one or more framework regions other than the framework regions flanking the CDR has been substituted or deleted, (v) one or more amino acid residues has been added in one or more framework regions other than the framework regions flanking said CDR, or (vi) a combination of (iv) and (v).

It is also an object of the invention to provide variants in which the CDR region has added or substituted therein at least one amino acid sequence which is heterologous to the CDR and the flanking framework regions, wherein the heterologous sequence is optionally capable of binding to a target sequence or molecule, and wherein the heterologous sequence is a MUC-1-binding portion of an anti-mucin monoclonal antibody. Preferably the variant includes a disrupted intrachain disulfide bond. Again, the construct has (i) one or more amino acid residues in one or more of the flanking framework regions may be substituted or deleted, (ii) one or more amino acid residues may be added in one or more of the flanking framework regions, or (iii) a combination of (i) and (ii); (i) one or more amino acid residues in one or more framework regions other than the framework regions flanking the CDR may be substituted or deleted, (ii) one or more amino acid residues may be added in one or more framework regions other than the framework regions flanking the CDR, or (iii) a combination of (i) and (ii), or (i) one or more amino acid residues in one or more of the flanking framework regions may be substituted or deleted, (ii) one or more amino acid residues may be added in one or more of the flanking framework regions, (iii) a combination of (i) and (ii); and (iv) one or more amino acid residues in one or more framework regions other than the framework regions flanking the CDR may be substituted or deleted, (v) one or more amino acid residues may be added in one or more framework regions other than the framework regions flanking the CDR, or (vi) a combination of (iv) and (v).

Preferably, the invention provides a construct containing a sequence encoding the variable region of an anti-mucin monoclonal antibody, preferably the anti-MUC-1 monoclonal antibody HMGF-1. In a specific embodiment, the construct of the present invention includes a sequence encoding the heavy and light chain variable region of HMFG-1, which optionally binds to the MUC-1 antigen through interactions of the CDR sequences of the HMFG-1 and MUC-1.

It is also an object of the invention to provide molecules including the variants described herein. The molecules can include one or more constant domains from an immunoglobulin; a second variable domain associated with the variant such as, for example, a variable domain of a heavy chain is associated with a variable domain of a light chain in an immunoglobulin; and a second variable domain associated with the variant, with one or more constant domains from immunoglobulins.

Also provided are immunoglobulins comprising a heavy chain and a light chain, wherein said heavy chain comprises a variant as described above and three constant domains from an immunoglobulin heavy chain, and the light chain comprises a second variable domain associated with the variant and a constant domain from an immunoglobulin light chain. Furthermore, the present invention provides immunoglobulins comprising a heavy chain and a light chain, wherein the light chain comprises a variant as described above and a constant domain from an immunoglobulin light chain, and the heavy chain comprises a second variable domain associated with said variant and three constant domains from an immunoglobulin heavy chain.

Isolated nucleic acids encoding these variants, molecules, and immunoglobulins are also provided, as are cells containing these nucleic acids. Recombinant non-human hosts containing these nucleic acids are also provided. Pharmaceutical compositions comprising a therapeutically or prophylactically effective amount of the variants, molecules or immunoglobulins and pharmaceutically acceptable carriers are also provided.

The invention further provides vaccine compositions that comprise an amount of the immunoglobulin construct effective to elicit an anti-idiotype response against mucin, particularly MUC-1. These compositions may further include a pharmaceutically acceptable carrier or excipient and an adjuvant.

Preferably, a synthetic antibody comprises one or more sequences from one or more CDRs of monoclonal antibody HMFG-1, preferably inserted in the corresponding CDR of the synthetic antibody having a disrupted intrachain disulfide bond in one or both variable domains. A vaccine composition, as set forth above, comprises this synthetic antibody.

In a further embodiment, it is an object of the invention to provide a nucleic acid encoding this synthetic antibody. The invention also furnishes vaccine compositions comprising the nucleic acid encoding the synthetic antibody in an amount effective to produce sufficient amounts to elicit a mucin-specific anti-idiotype antibody response. These compositions may further include a pharmaceutically acceptable carrier or excipient and an adjuvant.

Also encompassed are expression vectors, in which the nucleic acid is operably associated with an expression control sequence. The invention extends to host cells transfected or transformed with the expression vector. The synthetic construct or nucleic acid can be produced by isolating it from the host cells grown under conditions that permit production of the nucleic acid or expression of the synthetic antibody.

The pharmaceutical and vaccine compositions of the invention can be administered to a subject to elicit anti-mucin anti-idiotypic antibodies, particularly Ab2 antibodies, targeted against mucin, particularly MUC-1, bearing tumor cells.

### SUMMARY OF THE INVENTION

Thus, the invention provides a variant of an immunoglobulin variable domain, said immunoglobulin variable domain comprising (A) at least one CDR region and (B) framework regions flanking said CDR, said variant comprising:

(a) said CDR region having added or substituted therein at least one binding sequence; and

(b) said flanking framework regions,

wherein said binding sequence is heterologous to said CDR and is a binding sequence from a CDR of an anti-mucin monoclonal antibody, and wherein the variable domain lacks an intrachain disulfide bond.

Preferably, the invention provides a construct containing a sequence encoding the variable region of an anti-mucin monoclonal antibody, preferably the anti-MUC-1 monoclonal antibody HMGF-1. In a specific embodiment, the construct of the present invention includes a sequence encoding the heavy and light chain variable region of HMFG-1, which optionally binds to the MUC-1 antigen through interactions of the CDR sequences of the HMFG-1 and MUC-1.

In a preferred embodiment, the variant described above has (i) one or more amino acid residues in one or more of said flanking framework regions substituted or deleted, (ii) one or more amino acid residues added in one or more of said flanking framework regions, or (iii) a combination of (i) and (ii). Alternatively, the variant described above has (i) one or more amino acid residues in one or more framework regions other than said framework regions flanking said CDR substituted or deleted, (ii) one or more amino acid residues added in one or more framework regions other than said framework regions flanking said CDR, or (iii) a combination of (i) and (ii). The variant as described above may also include (i) one or more amino acid residues in one or more of said flanking framework regions substituted or deleted, (ii) one or more amino acid residues added in one or more of said flanking framework regions, or (iii) a combination of (i) and (ii); and wherein (iv) one or more amino acid residues in one or more framework regions other than said framework regions flanking said CDR has been substituted or deleted, (v) one or more amino acid residues has been added in one or more framework regions other than said framework regions flanking said CDR, or (vi) a combination of (iv) and (v).

The present invention also provides a variant of an immunoglobulin variable domain, said immunoglobulin variable domain comprising (A) at least one CDR region and (B) framework regions flanking said CDR, said variant comprising:

(a) said CDR region having added or substituted therein at least one amino acid sequence which is heterologous to said CDR and

(b) said flanking framework regions,

wherein said heterologous sequence is a CDR of an anti-mucin monoclonal antibody, and wherein the variable domain lacks an intrachain disulfide bond.

The variant describe above may have (i) one or more amino acid residues in one or more of said flanking framework regions substituted or deleted, (ii) one or more amino acid residues added in on or more of said flanking framework regions, or (iii) a combination of (i) and (ii). In addition, the variant described above may have (i) one or more amino acid residues in one or more framework regions other than said framework regions flanking said CDR substituted or deleted, (ii) one or more amino acid residues added in one or more framework regions other than said framework regions flanking said CDR, or (iii) a combination of (i) and (ii). Additionally, the variant may include (i) one or more amino acid residues in one or more of said flanking framework regions has been substituted or deleted, (ii) one or more amino acid residues has been added in one or more of said flanking framework regions, (iii) a combination of (i) and (ii); and wherein (iv) one or more amino acid residues in one or more framework regions other than said framework regions flanking said CDR has been substituted or deleted, (v) one or more amino acid residues has been added in one or more framework regions other than said framework regions flanking said CDR, or (vi) a combination of (iv) and (v).

The invention also provides a variant as described above including one or more of the following:

(1) said mucin is MUC-1;

(2) said monoclonal antibody is HMFG-1;

(3) said CDR of an anti-mucin monoclonal antibody is more than one CDR;

(4) said heterologous sequence is a CDR of a heavy chain variable region;

(5) said heterologous sequence is a CDR of a light chain variable region;

(6) said heterologous sequence comprises an amino acid sequence selected from the group consisting of AYWIE (SEQ ID NO: 1) in CDR1, EILPGSNNSRYNEKFKG (SEQ ID NO: 2) in CDR2, and SYDFAWFAY (SEQ ID NO: 3) in CDR3 of a human heavy chain variable region, and KSSQSLLYSSNQKIYLA (SEQ ID NO: 4) in CDR1, WASTRES (SEQ ID NO: 5) in CDR2, and QQYYRYPRT (SEQ ID NO: 6) in CDR3 of a human light chain variable region; and

(7) said CDR region is CDR 1, CDR 2 or CDR 3.

In addition, the invention provides a variant as described above which is an antibody.

The invention also provides molecules including the variant(s) described above, as well as molecules further comprising (1) one or more constant domains from an immunoglobulin; (2) a second variable domain linked to said variant; (3) a second variable domain linked to said variant, and one or more constant domains from an immunoglobulin.

Further, the invention provides a molecule as described above wherein said heterologous sequence is a CDR from monoclonal antibody HMFG-1, and optionally, the CDR region is selected from the group consisting of CDR 1, CDR 2, and CDR3.

The invention further provides a molecule as described above which is an antibody. The molecule described above may optionally comprise an amino acid sequence as depicted in SEQ ID NO: 7 or SEQ ID NO: 8, or both.

The invention further provides a molecule as described above which is derived from a human antibody, a chimeric or a humanized antibody.

The invention also provides an immunoglobulin comprising a heavy chain and a light chain, wherein said heavy chain comprises a variant as described above and three constant domains from an immunoglobulin heavy chain, and said light chain comprises a second variable domain associated with said variant and a constant domain from an immunoglobulin light chain.

In addition, the invention provides an immunoglobulin comprising a heavy chain and a light chain, wherein said light chain comprises a variant as described above and a constant domain from an immunoglobulin light chain, and said heavy chain comprises a second variable domain associated with said variant and three constant domains from an immunoglobulin heavy chain.

Moreover, the invention provides an isolated nucleic acid encoding a variant or immunoglobulin as described above, and a cell and/or recombinant non-human host containing such nucleic acid.

The invention further provides a vaccine composition comprising a therapeutically or prophylactically effective amount of a variant, molecule, immunoglobulin, or nucleic acid as described above and an adjuvant.

Still further, the invention provides a method of treating or preventing an adenocarcinoma tumor in a subject in need of such treatment or prevention, said method comprising administering to said subject a disease treating or preventing effective amount of a variant, immunoglobulin, or nucleic acid as described above.

### DESCRIPTION OF THE DRAWINGS

FIGS. 1A and 1B. Consensus amino acid sequences of (A) the heavy chain variable region (SEQ ID NO: 7) and (B) the light chain variable region (SEQ ID NO: 8).

FIG. 2. Diagram of PCR knitting.

FIG. 3. DNA sequence of HMFG-1 heavy chain variable region gene (Ala).

FIG. 4. DNA Sequence of HMFG-1 light chain variable region gene (Ala).

FIG. 5. Strategy for assembling variable region.

FIGS. 6A and 6B. Binding of HMFG-1 synthetic antibody/vaccines to OVCAR cells. OVCAR cells were incubated with human (A) or murine (B) HMFG-1 synthetic antibody/vaccines and control antibodies (IgG1 and consensus Ab). The binding of the HMFG-1 synthebody/vaccines and control Abs to the cells was probed with FITC-labeled goat-anti-human or mouse IgG and evaluated by flow cytometry. The results are expressed as mean of percentage of positive cells±S.D. Several versions of the vaccine were tested. C/A version: The cysteine involved in the formation of intra-chain disulfide bond in the CDR region of the light chain was replaced by alanine. A/C version: The cysteine involved in the formation of intra-chain disulfide bond in the CDR region of the heavy chain was replaced by alanine. C/C version: No cysteine replacement was performed.

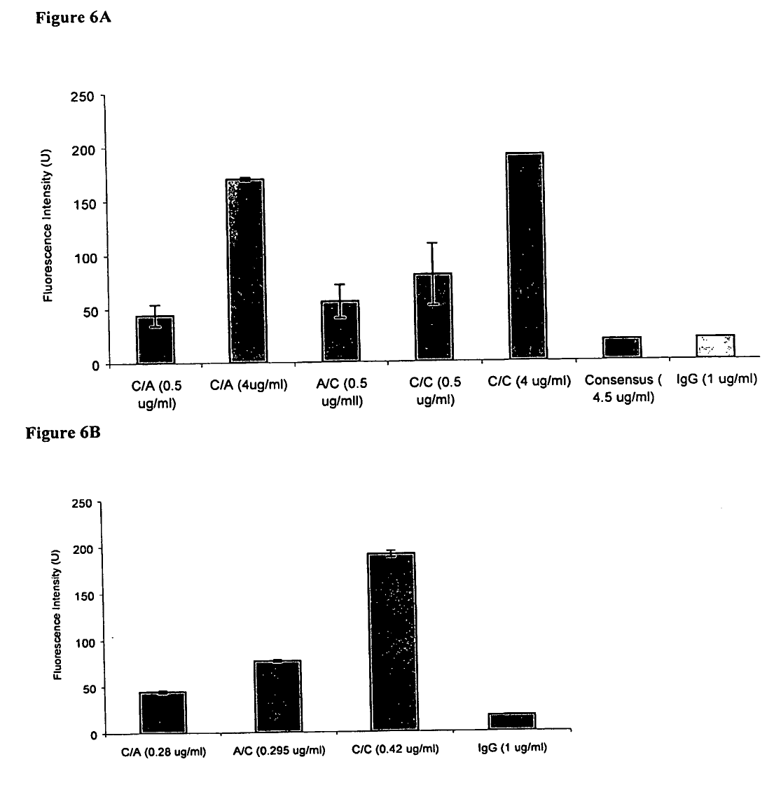

### DETAILED DESCRIPTION

The present invention provides an approach to elicit an anti-tumor immune response against tumors bearing mucins. The invention involves introducing the variable domains from an anti-mucin antibody, e.g., the anti-MUC1 antibodyHMFG-1, into a synthetic construct designed to elicit an anti-idiotypic immune response. In particular, Ab2 antibodies in an anti-idiotype pathway target mucin-bearing tumor cells for destruction.

Thus, in one aspect, the invention provides a synthetic antibody that contains variable domain sequences from an anti-mucin monoclonal antibody, preferably with a disrupted intrachain disulfide bond in the variable domain. The invention further provides vaccine compositions that comprise an amount of the synthetic antibody effective to elicit an anti-mucin Ab2 anti-idiotype response in vivo, a pharmaceutically acceptable carrier or excipient, and optimally an adjuvant.

Recombinant nucleic acids, particularly DNA molecules, provide for efficient expression of the foregoing constructs. In one aspect of this embodiment, the invention provides a nucleic acid encoding the synthebody. Also encompassed are expression vectors in which the nucleic acid is operably associated with an expression control sequence. The invention extends to host cells transfected or transformed with the expression vector. The construct can be produced by isolating it from the host cells grown under conditions that permit expression of the synthebody.

The invention also furnishes a vaccine composition comprising a vector that expresses the construct in an amount effective to produce sufficient construct to elicit an anti-mucin Ab2 response.

The present invention is based on construction of synthetic modified antibodies containing HMFG-1 variable domain sequences. Using oligodeoxyribonucleotides, heavy and light chain variable region genes were constructed to contain the CDR sequences from HMFG-1. The engineered variable region was then inserted into a mammalian expression vector containing the appropriate constant regions. A vector containing both light and heavy chains was transfected into a mammalian cell and the synthetic antibody was expressed. The synthetic modified antibodies were assayed for their ability to bind to mucin.

The primary activity of the anti-mucin synthetic construct is to elicit an anti-tumor anti-idiotype immune response in order to cause tumor regression, increase the time to relapse, and decrease mortality. The slower clearance seen with antibodies and other immunoglobulin superfamily proteins combined with an enhanced ability to elicit an anti-idiotypic antibody response gives the anti-mucin constructs an advantage over mucin-specific monoclonal antibodies for cancer therapy in vivo.

The term “construct” refers to the variant of a variable domain of an immunoglobulin superfamily protein, including molecules comprising such variants, described herein. The immunoglobulin superfamily is well known, and includes antibody/B-cell receptor proteins, T lymphocyte receptor proteins, and other proteins mentioned infra (see, Paul, Fundamental Immunology, 3^{rd} Ed.). In a preferred embodiment, the constructs of the invention are prepared by cloning the variable domain sequences for both the light and heavy chains. Preferably, the constructs of the invention comprise the complete murine variable domain from the HMFG-1 antibody.

A synthetic antibody is a specific example of a construct of the invention that includes an antibody variable region. It may also include regions corresponding to an antibody constant region or regions, or be associated with one or more other immunoglobulin family polypeptides, such as an antibody Fv heterodimer, an antibody tetramer, a T lymphocyte receptor heterodimer, etc. Embodiments described below are illustrative of the variants and molecules of the present invention in that the variants are included in synthetic antibodies and synthetic antibodies are a type of molecule that includes the variants. The term “synthetic antibody” thus refers to an illustrative example of a type of construct of the invention.

The term “heterologous” refers to a combination of elements not naturally occurring in a particular locus. For example, heterologous DNA refers to DNA not naturally located in the cell or in a particular chromosomal site of the cell. A heterologous expression regulatory element is such an element operatively associated with a different gene than the one it is operatively associated with in nature. In the context of the present invention, a construct coding sequence is heterologous to the vector DNA in which it is inserted for cloning or expression and it is heterologous to a host cell containing such a vector in which it is expressed, e.g., a CHO cell. Moreover, the constructs of the present invention contain a heterologous DNA, amino acid, or binding sequence.

The “heterologous amino acid (or binding) sequence” (also “binding to sequence”) refers to the desired binding segment of a polypeptide, e.g., the portion of a polypeptide (protein or peptide) that binds to a receptor. As used in this application, the term refers to the sequence of a CDR from an anti-mucin antibody.

An anti-mucin antibody is any monoclonal antibody that immunospecifically binds to a mucin, such as MUC1. Immunospecific binding means that the antibody binds the antigen with greater affinity than any or most any other antigen, such as a cross-reacting antigen. Anti-mucin antibodies from which one or more variable domain sequences are drawn to generate a construct of the invention are termed herein “parent” anti-mucin antibodies. Anti-mucin antibodies preferably are specific for MUC-1; HMFG-1 and HMFG-2 are examples of such anti-MUC-1 antibodies (Lalani, et al., J. Biol. Chem. 1991, 266: 15420-15426; Burchell and Taylor-Papadimitriou, Epithelial Cell Biol. 1993, 2: 155-162).

The term “CDR” refers to a part of the variable region of an immunoglobulin family protein that confers binding specificity, e.g., antibody specificity for antigen. In antibodies, CDRs are highly variable and accessible.

The term “framework region” refers to the part of the modified immunoglobulin molecule corresponding to an antibody framework region, as defined in the art. Sequences flanking the CDR are termed herein “framework regions of a variable region”.

The term “flanked” and “flanking” refers to the amino acids that are connected to or are connected by spacing amino acids to the protein sequence of the CDR. “Spacing amino acids” (or a “spacer group”) are amino acids that are not found in the native framework sequence or the CDR or the substituted sequence, nor do they independently confer any binding activity on the modified variable region. They may be included to preserve or ensure a proper variable region conformation and orientation of the CDR.

The term “vaccine” refers to a composition (protein or vector; the latter may also be loosely termed a “DNA vaccine”, although RNA vectors can be used as well) that can be used to elicit protective immunity in a recipient. It should be noted that to be effective, a vaccine of the invention can elicit immunity in a portion of the population, as some individuals may fail to mount a robust or protective immune response, or, in some cases, any immune response. This inability may stem from the individual's genetic background or because of an immunodeficiency condition (either acquired or congenital) or immunosuppression (e.g., treatment with immunosuppressive drugs to prevent organ rejection or suppress an autoimmune condition). Efficacy can be established in animal models.

The term “immunotherapy” refers to a treatment regimen based on activation of a tumor-specific immune response. A vaccine can be one form of immunotherapy. Immunotherapy differs from vaccination in that the former usually refers to prophylaxis, while therapy can improve the course of a disease. The present invention permits both.

The term “protect” is used herein to mean prevent or treat, or both, as appropriate, development of a mucin-bearing cancer, e.g., an adenocarcinoma, in a subject. Thus, prophylactic administration of the vaccine can protect the recipient subject at risk of developing such a carcinoma, e.g., as determined from family history.

The phrase “pharmaceutically acceptable”, whether used in connection with the pharmaceutical compositions of the invention or vaccine compositions of the invention, refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, cholera toxin, BCG (bacille Calmette-Guerin) and Corynebacterium parvum. Preferably, the adjuvant is pharmaceutically acceptable.

The term “about” or “approximately” will be known to those skilled in the art in light of this disclosure. Preferably, the term means within 20%, more preferably within 10%, and more preferably still within 5% of a given value or range. Alternatively, especially in biological systems, the term “about” preferably means within about a log (i.e., an order of magnitude), preferably within a factor of two of a given value, depending on how quantitative the measurement.

### Molecular Biology—Definitions

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene”, also called a “structural gene” means a DNA sequence that codes for or corresponds to a particular sequence of amino acids which comprise all or part of one or more proteins, and may or may not include regulatory DNA sequences, such as promoter sequences, that determine for example the conditions under which the gene is expressed. The transcribed region of a gene can include 5′- and 3′-untranslated regions (UTRs) and introns in addition to the translated (coding) region.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of or “operably associated with” transcriptional and translational control sequences in a cell. RNA polymerase transcribes the coding sequence into mRNA that is then trans-RNA spliced (if it contains introns) and translated into the protein encoded by the coding sequence.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a mRNA or a protein. The expression product itself, e.g. the resulting mRNA or protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell. “Conditions that permit expression” in vitro are culture conditions of temperature (generally about 37° C.), humidity (humid atmosphere), carbon dioxide concentration to maintain pH (generally about 5% CO₂ to about 15% CO₂), pH (generally about 7.0 to 8.0, preferably 7.5), and culture fluid components that depend on host cell type. In vivo, the conditions that permit expression are primarily the health of the non-human transgenic animal that depends on adequate nutrition, water, habitation, and environmental conditions (light-dark cycle, temperature, humidity, noise level). In either system, expression may depend on a repressor or inducer control system, as well known in the art.

The term “transfection” means the introduction of a “foreign” (i.e., extrinsic or extracellular) gene, DNA or RNA sequence into a host cell, so that the host cell will express the introduced gene or sequence to produce a desired substance, typically a protein or enzyme encoded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transfected” and is a “transfectant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g., a foreign gene) can be introduced into a host cell, so as to transfect the host and promote expression (e.g., transcription and translation) of the introduced sequence. Vectors include plasmids, phages, viruses, etc.; they are discussed in greater detail below.

Vectors typically comprise the DNA of a transmissible agent into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA segment that can be inserted into a vector or into another piece of DNA at a defined restriction site. Preferably, a cassette is an “expression cassette” in which the DNA is a coding sequence or segment of DNA that codes for an expression product that can be inserted into a vector at defined restriction sites. The cassette restriction sites generally are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid” that generally is a self-contained molecule of double-stranded DNA, usually of bacterial origin, that can readily accept additional (foreign) DNA and which can be readily introduced into a suitable host cell. A plasmid vector often contains coding DNA and promoter DNA and has one or more restriction sites suitable for inserting foreign DNA. A large number of vectors, including plasmid and fungal vectors, have been described for replication and/or expression in a variety of eukaryotic and prokaryotic hosts. Non-limiting examples include pKK plasmids (Amersham Pharmacia Biotech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme. Host cells can further be used for screening or other assays, as described infra. The host cell may be found in vitro, i.e., in tissue culture, or in vivo, i.e., in a microbe, plant or animal.

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. In a specific embodiment, the synthetic antibody is expressed in COS-1 or CHO cells. Other suitable cells include NSO cells, HeLa cells, 293T (human kidney cells), mouse primary myoblasts and NIH 3T3 cells.

The terms “mutant” and “mutation” mean any detectable change in genetic material, e.g., DNA, or any process, mechanism, or result of such a change. This includes gene mutations, in which the structure (e.g., DNA sequence) of a gene is altered, any gene or DNA arising from any mutation process, and any expression product (e.g., protein or enzyme) expressed by a modified gene or DNA sequence. The term “variant” may also be used to indicate a modified or altered gene, DNA sequence, enzyme, cell, etc., i.e., any kind of mutant.

“Sequence-conservative variants” of a polynucleotide sequence are those in which a change of one or more nucleotides in a given codon position results in no alteration in the amino acid encoded at that position.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, more preferably at least 85%, and even more preferably at least 90%, and that has the same or similar properties or functions as the native or parent protein or enzyme to which it is compared.

As used herein, the term “oligonucleotide” refers to a nucleic acid, generally of at least 10, preferably at least 15, and more preferably at least 20 nucleotides, preferably no more than about 100 nucleotides, that is hybridizable to a genomic DNA molecule, a cDNA molecule, or an mRNA molecule having a sequence of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of the synthetic antibody, or to detect the presence of nucleic acids encoding the synthetic antibody. In a further embodiment, an oligonucleotide of the invention can form a triple helix with a synthetic antibody-encoding DNA molecule, e.g., for purification purposes. Generally, oligonucleotides are prepared synthetically, preferably on a nucleic acid synthesizer. Accordingly, oligonucleotides can be prepared with non-naturally occurring phosphoester analog bonds, such as thioester bonds, etc.

### Constructs

The constructs of the invention can be derived from any type of immunoglobulin molecule, for example, but not limited to, antibodies, T lymphocyte receptors, cell-surface adhesion molecules such as the co-receptors CD4, CD8, CD19, and the invariant domains of MHC molecules. In a preferred embodiment of the invention, the construct is derived from an antibody that can be any class of antibody, e.g., an IgG, IgE, IgM, IgD or IgA, preferably, the antibody is an IgG. Such antibodies may be in membrane bound (B cell receptor) or secreted form, preferably secreted. Additionally, the antibody may be of any subclass of the particular class of antibodies. In another specific embodiment, the construct is derived from a T lymphocyte receptor.

CDR-grafted variable region genes have been constructed by various methods such as site-directed mutagenesis as described in Jones et al., Nature 1986, 321:522; Riechmann et al., Nature 1988, 332:323; in vitro assembly of entire CDR-grafted variable regions (Queen et al., Proc. Natl. Acad. Sci. USA 1989, 86:10029); and the use of PCR to synthesize CDR-grafted genes (Daugherty et al., Nucleic Acids Res. 1991, 19:2471). CDR-grafted antibodies are generated in which the CDRs of the murine monoclonal antibody are grafted onto the framework regions of a human antibody. Following grafting, most antibodies benefit from additional amino acid changes in the framework region to maintain affinity, presumably because framework residues are necessary to maintain CDR conformation, and some framework residues have been demonstrated to be part of the antigen combining site. Such CDR-grafted antibodies have been successfully constructed against various antigens, for example, antibodies against IL-2 receptor as described in Queen et al. (Proc. Natl. Acad. Sci. USA 1989, 86:10029), antibodies against cell surface receptors-CAMPATH as described in Riechmann et al. (Nature, 1988, 332:323); antibodies against hepatitis B in Co et al. (Proc. Natl. Acad. Sci. USA 1991, 88:2869); as well as against viral antigens of the respiratory syncitial virus in Tempest et al. (BioTechnology 1991, 9:267). Thus, in specific embodiments of the invention, the construct comprises a variable domain in which at least one of the framework regions has one or more amino acid residues that differ from the residue at that position in the naturally occurring framework region. The techniques employed in creating CDR-grafted antibodies can be adapted for use in constructs of the invention.

The heterologous amino acid sequence or sequences can be inserted into any one or more of the CDR regions of the variable domain variant, preferably into the corresponding CDR or CDRs of the variable domain variant. It is within the skill in the art to insert the CDRs from the parent anti-mucin antibody into different or corresponding CDRs of the variable domain or domains of the synthetic construct, and then screen the resulting modified constructs for the ability to bind to the mucin antigen or to elicit an appropriate anti-idiotypic antibody response. Thus, one can determine which CDR of CDRs optimally contain the CDRs from the parent anti-mucin antibody. In specific embodiments in which the construct is an antibody, a CDR or CDRs of either the heavy or light chain variable region, or both, are modified to contain the heterologous amino acid sequence. In another specific embodiment, the construct contains a variable domain in which the first, second, and third CDR of the heavy chain variable region or the first, second, and third CDR of the light chain variable region, and preferably both, contain the amino acid sequence of the corresponding CDR from the parent anti-mucin antibody. Corresponding modifications are also contemplated for other immunoglobulin superfamily protein-derived constructs of the invention.

In specific embodiments of the invention, the heterologous amino acid sequence is either inserted into the CDR without replacing any of the amino acid sequence of the CDR itself or, alternatively, the binding site amino acid sequence replaces all or a portion of the amino acid sequence of the CDR.

Relative efficacy of a mucin-binding construct to generate an anti-mucin anti-idiotype response can be evaluated by direct binding assays, such as ELISA, Western blotting, direct binding to mucin-containing cells (that can be detected by radiolabelling or fluorescence labeling) and the like; inhibition assays, e.g., with labeled soluble mucin; and functional assays, including tumor clearance in in vivo animal models of adenocarcinoma.

The constructs of the invention may also be further modified in any way known in the art, e.g., for the modification of antibodies. The modification may be such that the binding activity of the parent molecule is retained, e.g., the modification results in a construct retaining about 100% of the parent molecule's binding activity to the target antigen, preferably, at least 75% activity is retained, more preferably at least 50% activity is retained, and most preferably at least 25% activity is retained. Alternatively, the constructs of the invention do not bind the particular binding partner, e.g., less than 50%, and more preferably less than 25% of the activity of the parent molecule is retained in the construct of the invention.

In particular, the constructs of the invention may have one or more amino acid substitutions, deletions, or insertions besides the insertion into or replacement of CDR sequences with the binding sequence. Such amino acid substitutions, deletions, or insertions can be any substitution, deletion, or insertion that does not prevent the specific binding of the construct to the target. For example, such amino acid substitutions include substitutions of functionally equivalent amino acid residues. One or more amino acid residues can be substituted by another amino acid of a similar polarity that acts as a functional equivalent resulting in a silent alteration. Substitutes for an amino acid may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Additionally, one or more amino acid residues can be substituted by a nonclassical amino acid or chemical amino acid analogs, introduced as a substitution or addition into the immunoglobulin sequence. Non-classical amino acids include but are not limited to the D-isomers of the common amino acids, alpha-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, beta-alanine, fluoro-amino acids, designer amino acids such as beta-methyl amino acids, C-alpha-methyl amino acids, N-alpha-methyl amino acids, and amino acid analogs in general. Furthermore, the amino acid can be D (dextrorotary) or L (levorotary).

### Anti-Idiotype Constructs

To ensure a robust anti-idiotype response, the construct is further modified to enhance its ability to elicit an anti-idiotype response, for example, as described in PCT Publication No. WO 99/25379. Such modifications are made to reduce the conformational constraints on a variable domain, e.g., by removing or reducing intrachain disulfide bonds. Specifically, the construct is further modified such that one or more variable region cysteine residues that form disulfide bonds are replaced with an amino acid residue that does not have a sulfhydryl group.

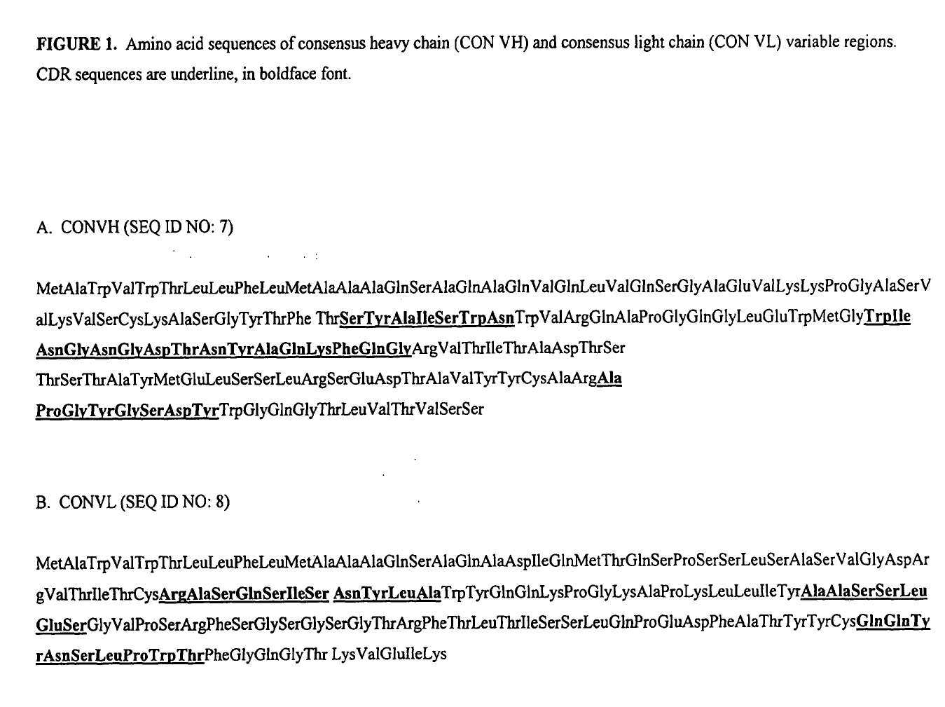

Identifying the cysteine residues that form a disulfide bond in a variable region of a particular antibody can be accomplished by any method known in the art. For example, but not by way of limitation, it is well known in the art that the cysteine residues that form intrachain disulfide bonds are highly conserved among antibody classes and across species. Thus, the cysteine residues that participate in disulfide bond formation can be identified by sequence comparison with other antibody molecules in which it is known which residues form a disulfide bond (for example the consensus sequences provided in FIGS. 1A and 1B, or those described in Kabat et al., 1991, Sequences of Proteins of Immunological Interest, 5^{th} Ed., U.S. Department of Health and Human Services, Bethesda, Md.).

Notably, for most antibody molecules, the cysteine residues that form the intrachain disulfide bonds are residues at positions 23 and 88 of the light chain variable domain and residues at positions 22 and 92 of the heavy chain variable domain. The position numbers refer to the residue corresponding to that residue in the consensus sequences as defined in Kabat et al., supra, i.e. the “Kabat equivalent” or as indicated in the heavy and light chain variable region sequences depicted in FIGS. 1A and 1B, respectively (as determined by aligning the particular antibody sequence with the consensus sequence of the heavy or light chain variable region sequence depicted in FIGS. 1A and 1B).

Accordingly, in one embodiment of the invention, the construct is further modified such that the residues at positions 23 and/or 88 of the light chain as identified by Kabat et al., supra, or their equivalent, are substituted with an amino acid residue that does not contain a sulfhydryl group; and/or the residues at positions 22 and/or 92 are of the heavy chain as identified by Kabat et al., supra, or their equivalent, are substituted with an amino acid residue that does not contain a sulfhydryl group. As used in reference to intrachain disulfides, equivalents of the cysteines at the Kabat positions provided above are intrachain-forming cysteine residues at homologous positions in the immunoglobulin domain of an immunoglobulin superfamily protein molecule.

The amino acid residue that substitutes for the disulfide bond forming cysteine residue is any amino acid residue that does not contain a sulfhydryl group, e.g., alanine, arginine, asparagine, aspartate (or aspartic acid), glutamine, glutamate (or glutamic acid), glycine, histidine, isoleucine, leucine, lysine, phenylalanine, proline, serine, threonine, tryptophan, tyrosine or valine. In a preferred embodiment, the cysteine residue is replaced with a glycine, serine, threonine, tyrosine, asparagine, or glutamine residue, most preferably with an alanine residue.

Additionally, the disulfide bond forming cysteine residue may be replaced by a nonclassical amino acid or chemical amino acid analog, such as those listed supra, that does not contain a sulfhydryl group; or it may be chemically modified by reaction with the sulfhydryl to preclude disulfide bond formation.

In specific embodiments, the substitution of the disulfide bond forming residue is in the heavy chain variable region or the light chain variable region, or both the heavy chain and light chain variable regions. In other specific embodiments, one of the residues that forms a particular disulfide bond is replaced (or deleted) or, alternatively, both residues that form a particular disulfide bond may be replaced (or deleted).

In a preferred embodiment, the constructs of the invention exhibit reduced binding, e.g., less than 75%, preferably less than 50%, and most preferably less than 25% of the binding activity of the parent molecule. Without wishing to be bound by any theory, such constructs may be more likely to elicit an anti-idiotypic response.

### Immunoglobulin Fragment Constructs

As noted above, fragments of an immunoglobulin family protein can be modified to create a construct. For example, such fragments include but are not limited to: F(ab′)₂ fragments that contain the variable regions of both the heavy and the light chains, the light chain constant region and the CH1 domain of the heavy chain, which fragments can be generated by pepsin digestion of an antibody; Fab fragments generated by reducing the disulfide bonds of an F(ab′)₂ fragment (King et al., Biochem. J., 1992, 281:317); and Fv fragments, i.e., fragments that contain the variable region domains of both the heavy and light chains (Reichmann and Winter, J. Mol. Biol. 1988, 203:825; King et al., Biochem J. 1993, 290:723).

Thus, the present invention includes, but is not limited to, single chain antibodies (SCA) (U.S. Pat. No. 4,946,778; Bird, Science 1988, 242:423-426; Huston et al., Proc. Natl Acad. Sci. USA 1988, 85:5879-5883; and Ward et al., Nature 1989, 334:544-546). Single chain antibodies are formed by linking the heavy and light chain fragments of the Fv region via an amino acid bridge, resulting in a single chain polypeptide. Additionally, the invention also provides heavy chain and light chain dimers and diabodies.

### Preferred Immunoglobulin Family Proteins

The immunoglobulin molecule modified to generate the constructs is preferably a monoclonal antibody. The antibody that is modified may be a naturally occurring or previously existing antibody, or may be synthesized from known antibody consensus sequences, such as the consensus sequences for the light and heavy chain variable regions in FIGS. 1A and 1B, or any other antibody consensus or germline (i.e., unrecombined genomic sequences) sequences (e.g., those antibody consensus and germline sequences described in Kabat et al., 1991, Sequences of Proteins of Immunological Interest, 5^{th} edition, NIH Publication No. 91-3242, pp. 2147-2172).

The invention further provides constructs that are modified chimeric or humanized antibodies. A chimeric antibody is a molecule in which different portions of the antibody molecule are derived from different animal species, such as those having a variable region derived from a murine mAb and a constant region derived from a human immunoglobulin constant region. Techniques have been developed for the production of chimeric antibodies (Morrison et al., Proc. Natl. Acad. Sci. USA 1984, 81:6851-6855; Neuberger et al., Nature, 1984, 312:604-608; Takeda et al., Nature 1985, 314:452-454; International Patent Application No. PCT/GB85/00392) by splicing the genes from a mouse antibody molecule of appropriate antigen specificity together with genes from a human antibody molecule of appropriate biological activity. In a specific embodiment, the synthetic antibody is a chimeric antibody containing the variable domain of a non-human antibody and the constant domain of a human antibody.

In another embodiment, the construct is derived from a humanized antibody, in which the CDRs of the antibody are derived from an antibody of a non-human animal and the framework regions and constant region are from a human antibody (see, U.S. Pat. No. 5,225,539).

As noted above, the construct can be derived from a human monoclonal antibody. The creation of completely human monoclonal antibodies is possible through the use of transgenic mice. Transgenic mice in which the mouse immunoglobulin gene loci have been replaced with human immunoglobulin loci provide in vivo affinity-maturation machinery for the production of human immunoglobulins.

### Immunoglobulin Fusion Protein and Derivative Construct

In certain embodiments, the construct is created by fusing (joining) an immunoglobulin family protein to an amino acid sequence of another protein (or portion thereof, preferably an at least 10, 20, or 50 amino acid portion thereof) that is not the modified immunoglobulin, thereby creating a fusion (or chimeric) construct. Preferably, the fusion is via covalent bond (for example, but not by way of limitation, a peptide bond) at either the N-terminus or the C-terminus.

The construct may be further modified, e.g., by the covalent attachment of any type of molecule, as long as such covalent attachment does not prevent the development of an anti-mucin anti-idiotype response. For example, but not by way of limitation, the construct may be further modified, e.g., by glycosylation, acetylation, PEGylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc.

In specific embodiments of the invention, the construct is covalently linked to a therapeutic molecule, for example, to target the therapeutic molecule to a particular cell type or tissue, e.g., an accessory or antigen-presenting cell. The therapeutic molecule can be any type of therapeutic molecule known in the art, for example, but not limited to, a chemotherapeutic agent, a toxin, such as ricin, an antisense oligonucleotide, a radionuclide, an antibiotic, anti-viral, or anti-parasitic, etc.

### Structure of the Binding (Amino Acid) Sequence

One or more, including, all six CDRs contained in the consensus heavy and light chain variable region clones with the corresponding CDRs from an anti-mucin antibody, e.g., HMFG-1.

Heavy chain variable region of HMFG-1 (V_{H}):

| | | |
|---|---|---|
| CDR1 | AYWIE | (SEQ ID NO: 1) |
| CDR2 | EILPGSNNSRYNEKFKG | (SEQ ID NO: 2) |
| CDR3 | SYDFAWFAY | (SEQ ID NO: 3) |

Light chain variable region of HMFG-1 (V_{L}):

| | | |
|---|---|---|
| CDR1 | KSSQSLLYSSNQKIYLA | (SEQ ID NO: 4) |
| CDR2 | WASTRES | (SEQ ID NO: 5) |
| CDR3 | QQYYRYPRT | (SEQ ID NO: 6) |

The construct must minimally induce an Ab2 response, preferably also an Ab3 response.

### Methods of Producing the Constructs

Constructs can be produced by any method known in the art for the synthesis of immunoglobulins, in particular, by chemical synthesis or by recombinant expression, and are preferably produced by recombinant expression techniques.

Recombinant expression of constructs requires construction of a nucleic acid encoding the construct. Such an isolated nucleic acid that contains a nucleotide sequence encoding the construct can be produced using any method known in the art.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization, B. D. Hames & S. J. Higgins eds. (1985); Transcription And Translation, B. D. Hames & S. J. Higgins, eds. (1984); Animal Cell Culture, R. I. Freshney, ed. (1986); Immobilized Cells And Enzymes, IRL Press, (1986); B. Perbal, A Practical Guide To Molecular Cloning (1984); F. M. Ausubel et al. (eds.), Current Protocols in Molecular Biology, John Wiley & Sons, Inc. (1994).

### Construct Nucleic Acids

Accordingly, the invention provides nucleic acids that contain a nucleotide sequence encoding a construct of the invention.

A nucleic acid that encodes a construct may be assembled from chemically synthesized oligonucleotides (e.g., as described in Kutmeier et al., BioTechniques 1994, 17:242), that briefly, involves the synthesis of a set of overlapping oligonucleotides containing portions of the sequence encoding the protein, annealing and ligation of those oligonucleotides, and then amplification of the ligated oligonucleotides by PCR.

Accordingly, the invention provides a method of producing a nucleic acid encoding a construct, the method comprising: (a) synthesizing a set of oligonucleotides, the set comprising oligonucleotides containing a portion of the nucleotide sequence that encodes the construct and oligonucleotides containing a portion of the nucleotide sequence that is complementary to the nucleotide sequence that encodes the construct, and each of the oligonucleotides having overlapping terminal sequences with another oligonucleotide of the set, except for those oligonucleotides containing the nucleotide sequences encoding the N-terminal and C-terminal portions of the synthetic antibody; (b) allowing the oligonucleotides to hybridize or anneal to each other; and (c) ligating the hybridized oligonucleotides, such that a nucleic acid containing the nucleotide sequence encoding the synthetic synthebody is produced.

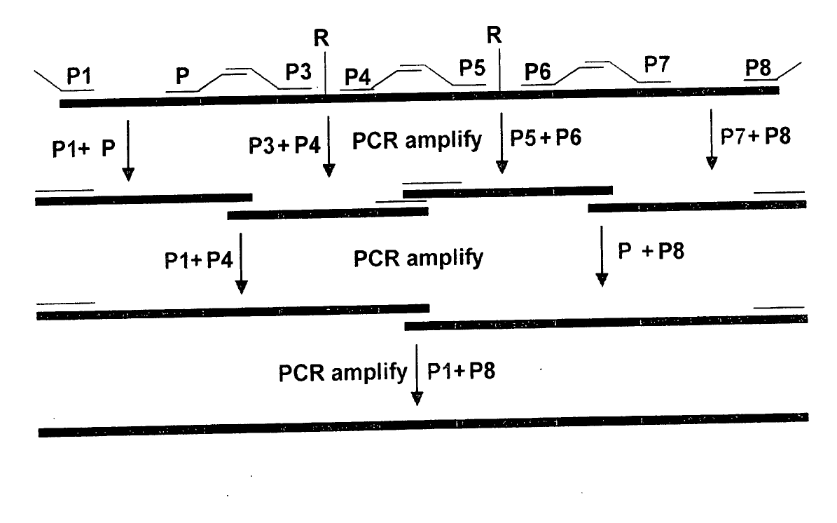

Another method for producing a nucleic acid encoding a construct is to modify nucleic acid sequences that encode an immunoglobulin superfamily molecule, e.g., an antibody molecule or at least the variable region thereof, using the “PCR knitting” approach (FIG. 2). In “PCR knitting”, nucleic acid sequences, such as the consensus variable region sequences shown in Example 1, are used as templates for a series of PCR reactions that result in the selective insertion of a nucleotide sequence that encodes the desired peptide sequence (in this example, the CDR of HMFG-1) into one or more CDRs of the variable domain. Oligonucleotide primers are designed for these PCR reactions that contain regions complementary to the framework sequences flanking the designated CDR at the 3′ends and sequences that encode the peptide sequence to be inserted at the 5′ends. In addition, these oligonucleotides contain approximately ten bases of complementary sequences at their 5′ends. These oligonucleotide primers can be used with additional flanking primers to insert the desired nucleotide sequence into the selected CDR as shown in FIG. 2 resulting in the production of a nucleic acid coding for the synthebody.

Alternatively, a nucleic acid containing a nucleotide sequence encoding a construct can be constructed from a nucleic acid containing a nucleotide sequence encoding, e.g., an antibody molecule, or at least a variable region of an antibody molecule. Nucleic acids containing nucleotide sequences encoding antibody molecules can be obtained either from existing clones of antibody molecules or variable domains or by isolating a nucleic acid encoding an antibody molecule or variable domain from a suitable source, preferably a cDNA library, e.g., an antibody DNA library or a cDNA library prepared from cells or tissue expressing a repertoire of antibody molecules or a synthetic antibody library (see, e.g., Clackson et al., Nature, 1991, 352:624; Hane et al., Proc. Natl. Acad. Sci. USA, 1997, 94:4937), for example, by hybridization using a probe specific for the particular antibody molecule or by PCR amplification using synthetic primers hybridizable to the 3′ and 5′ ends of the sequence.

The nucleic acid encoding the modified antibody optionally contains a nucleotide sequence encoding a leader sequence that directs the secretion of the synthetic antibody molecule.

### Construct Expression

Once a nucleic acid encoding a construct is obtained, it may be expressed or it may be introduced into a vector containing the nucleotide sequence encoding the constant region of the antibody (see, e.g., PCT Publications WO 86/05807 and WO 89/01036; and U.S. Pat. No. 5,122,464). Vectors containing the complete light or heavy chain for co-expression are available to allow the expression of a complete antibody molecule and are known in the art, for example, pMRRO10.1 and pGammal (see also, Bebbington, Methods a companion to Methods in Enzymology 1991, 2:136-145).

The expression vector can then be transferred to a host cell in vitro or in vivo by conventional techniques and the transfected cells can be cultured by conventional techniques to produce a construct of the invention. Specifically, once a variable region of the modified antibody has been generated, the modified antibody can be expressed, for example, by the method exemplified in the Examples (see also Bebbington, supra). For example, by transient transfection of the expression vector encoding a construct into COS cells, culturing the cells for an appropriate period of time to permit construct expression, and then taking the supernatant from the COS cells, which supernatant contains the secreted, expressed synthetic antibody.

The host cells used to express the recombinant construct of the invention may be either bacterial cells such as Escherichia coli, particularly for the expression of recombinant antibody fragments or, preferably, eukaryotic cells, particularly for the expression of recombinant immunoglobulin molecules. In particular, mammalian cells such as Chinese hamster ovary cells (CHO) or COS cells, used in conjunction with a vector in which expression of the construct is under control of the major intermediate early gene promoter element from human cytomegalovirus, is an effective expression system for immunoglobulins (Foecking et al., Gene 1986, 45:101; Cockett et al., BioTechnology 1990, 8:662).

A variety of host-expression vector systems may be utilized to express the construct coding sequences of the invention. Such host-expression systems represent vehicles by which the coding sequences of interest may be produced and subsequently purified, but may also be used to transform or transfect cells with the appropriate nucleotide coding and control sequences to produce the antibody product of the invention in situ. These systems include, but are not limited to, microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g, baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; mammalian cell systems (e.g., COS, CHO, BHK, 293, and 3T3 cells) harboring recombinant expression constructs containing promoters derived from the genome of mammalian cells (e.g., the metallothionein promoter) or from mammalian viruses (e.g., the adenovirus late promoter; the vaccinia virus 7.5K promoter); and transgenic animal systems, particularly for expression in milk (e.g., U.S. Pat. Nos. 5,831,141 and 5,849,992, which describe transgenic production of antibodies in milk; U.S. Pat. No. 4,873,316).

Expression of the construct may be controlled by any promoter/enhancer element known in the art, but these regulatory elements must be functional in the host selected for expression. Promoters that may be used to control gene expression include, but are not limited to, cytomegalovirus (CMV) promoter (U.S. Pat. Nos. 5,385,839 and 5,168,062), the SV40 early promoter region (Benoist and Chambon, Nature 1981, 290:304-310), the promoter contained in the 3′ long terminal repeat of Rous sarcoma virus (Yamamoto, et al. Cell, 1980, 22:787-797), the herpes thymidine kinase promoter (Wagner et al., Proc. Natl. Acad. Sci. USA 1981, 78:1441-1445), the regulatory sequences of the metallothionein gene (Brinster et al., Nature 1982, 296:3942); prokaryotic expression vectors such as the β-lactamase promoter (Villa-Komaroff, et al., Proc. Natl. Acad. Sci. USA 1978, 75:3727-3731), or the tac promoter (DeBoer, et al., Proc. Natl. Acad. Sci. USA 1983, 80:21-25); see also “Useful proteins from recombinant bacteria” in Scientific American 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region that is active in myeloid cells (Mogram et al., Nature 1985, 315:338-340; Kollias et al. 1986, Cell 46:89-94), hematopoietic stem cell differentiation factor promoters, erythropoietin receptor promoter (Maouche et al., Blood 1991, 15:2557), etc.

In bacterial systems, a number of expression vectors may be advantageously selected depending upon the use intended for the construct being expressed. For example, when a large quantity of such a protein is to be produced, for the generation of pharmaceutical compositions of a construct, vectors that direct the expression of high levels of readily purified fusion protein products may be desirable. Such vectors include, but are not limited to, the E. coli expression vector pUR278 (Ruther et al., EMBO J. 1983, 2:1791), in which the coding sequence may be ligated individually into the vector in frame with the lac Z coding region so that a fusion protein is produced; pIN vectors (Inouye & Inouye, Nucleic Acids Res. 1985, 13:3101-3109; Van Hleeke & Schuster, J. Biol. Chem. 1989, 264:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix of glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example, the polyhedrin gene) of the virus and placed under control of an AcNPV promoter (for example, the polyhedrin promoter).

In mammalian host cells, a number of viral-based and non-viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted into the adenovirus genome. Insertion in a non-essential region of the viral genome (e.g., region E1 or E3) will result in a recombinant virus that is viable and capable of expressing the construct in infected hosts (see, e.g., Logan and Shenk, Proc. Natl. Acad. Sci. USA 1984, 81:3655-3659). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 1987, 153:516-544).

Additionally, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include, but are not limited to, CHO, VERO, BHK, HeLa, COS, MDCK, 293, 3T3, W138.

For long-term, high-yield production of recombinant proteins, stable expression is preferred. For example, cell lines that stably express the antibody may be engineered. Rather than using expression vectors that contain viral origins of replication, host cells can be transformed with DNA controlled by appropriate expression control elements (e.g., promoter, enhancer sequences, transcription terminators, polyadenylation sites, etc.) and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form focithat, in turn, can be cloned and expanded into cell lines. This method may advantageously be used to engineer cell lines that express the antibody. Such engineered cell lines may be particularly useful in screening and evaluation of compounds that interact directly or indirectly with the antibody.

A number of selection systems may be used, including but not limited to the herpes simplex virus thymidine kinase (Wigler et al., Cell 1977, 11:223), hypoxanthine-guanie phosphoribosyltransferase (Szybalska and Szybalski, Proc. Natl. Acad. Sci. USA 1962, 48:2026), and adenine phosphoribosyltransferase (Lowy et al., Cell 1980, 22:817) genes can be employed in tk-, hgprt-, or aprt-cells, respectively. Also, antimetabolite resistance can be used as the basis of selection for the following genes: dhfr, which confers resistance to methotrexate (Wigler et al., Proc. Natl. Acad. Sci. USA 1980, 77:3567; O'Hare et al., Proc. Natl. Acad. Sci. USA 1981, 78:1527); gpt, which confers resistance to mycophenolic acid (Mulligan and Berg, Proc. Natl. Acad. Sci. USA 1981, 78:2072); neo, which confers resistance to the aminoglycoside G418 (Colberre-Garapin et al., J. Mol. Biol. 1981, 150:1); and hygro, which confers resistance to hygromycin (Santerre et al., Gene 1984, 30:147).

The expression levels of the construct can be increased by vector amplification (for a review, see Bebbington and Hentschel, The Use of Vectors Based on Gene Ampliflication for the Expression of Cloned Genes in Mammalian Cells in DNA Cloning, Vol. 3., Academic Press, New York, 1987). When a marker in the vector system expressing a construct is amplifiable, increases in the level of inhibitor present in the culture medium of the host cell will increase the number of copies of the marker gene. Since the amplified region is associated with the construct gene, production of the construct will also increase (Crouse et al., Mol. Cell. Biol. 1983, 3:257).

In a specific embodiment in which the construct is an antibody (immunoglobulin), the host cell may be co-transfected with two expression vectors of the invention, the first vector encoding a heavy chain derived polypeptide and the second vector encoding a light chain derived polypeptide. The two vectors may contain identical selectable markers that enable equal expression of heavy and light chain polypeptides. Alternatively, a single vector may be used that encodes both heavy and light chain polypeptides. In such situations, the light chain should be placed before the heavy chain to avoid an excess of toxic free heavy chain (Proudfoot, Nature 1986, 322:562; Kohler, Proc. Natl. Acad Sci. USA 1980, 77:2197). The coding sequences for the heavy and light chains may comprise cDNA or genomic DNA.

The invention provides a recombinant cell that contains a vector encoding a construct of the invention.

### Viral and Non-Viral Vectors

Preferred vectors, particularly for cellular assays in vitro and in vivo, are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant protein or polypeptide domain fragment thereof can be introduced in vivo, ex vivo, or in vitro using a viral vector or through direct introduction of DNA. Expression in targeted tissues can be affected by targeting the transgenic vector to specific cells, such as with a viral vector or a receptor ligand, or by using a tissue-specific promoter, or both. Targeted gene delivery is described in PCT Publication No. WO 95/28494.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques 1992, 7:980-990). Preferably, the viral vectors are replication-defective, that is, they are unable to replicate autonomously in the target cell. Preferably, the replication defective virus is a minimal virus, i.e., it retains only the sequences of its genome that are necessary for encapsidating the genome to produce viral particles.

DNA viral vectors include an attenuated or defective DNA virus, such as but not limited to, herpes simplex virus (HSV), papillomavirus, Epstein Barr virus (EBV), adenovirus, adeno-associated virus (AAV), and the like. Defective viruses that entirely or almost entirely lack viral genes are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV 1) vector (Kaplitt et al., Molec. Cell. Neurosci. 1991, 2:320-330), defective herpes virus vector lacking a glyco-protein L gene, or other defective herpes virus vectors (PCT Publication Nos. WO 94/21807 and WO 92/05263); an attenuated adenovirus vector, such as the vector described by Stratford-Perricaudet et aL (J. Clin. Invest. 1992, 90:626-630; see also La Salle et al., Science 1993, 259:988-990); and a defective adeno-associated virus vector (Samulski et al., J. Virol., 1987, 61:3096-3101; Samulski et al., J. Virol. 1989, 63:3822-3828; Lebkowski et al., Mol. Cell. Biol. 1988, 8:3988-3996).

Various companies produce viral vectors commercially, including, but not limited to, Avigen, Inc. (Alameda, Calif.; AAV vectors), Cell Genesys (Foster City, Calif.; retroviral, adenoviral, AAV, and lentiviral vectors), Clontech (retroviral and baculoviral vectors), Genovo, Inc. (Sharon Hill, Pa.; adenoviral and AAV vectors), Genvec (France; adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

Adenovirus vectors. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type 2 or type 5 human adenoviruses (Ad 2 or Ad 5) or adenoviruses of animal origin (see PCT Publication No. WO 94/26914). Those adenoviruses of animal origin that can be used within the scope of the present invention include adenoviruses of canine, bovine, murine (example: Mavl, Beard et al., Virology 1991, 180:257-65), ovine, porcine, avian, and simian (example: SAV) origin. Preferably, the adenovirus of animal origin is a canine adenovirus, more preferably a CAV2 adenovirus (e.g., Manhattan or A26/61 strain, ATCC VR-800, for example). Various replication defective adenovirus and minimum adenovirus vectors have been described (PCT Publication Nos. WO 94/26914, WO 95/02697, WO 94/28938, WO 94/28152, WO 94/12649, WO 95/02697, WO 96/22378). The replication defective recombinant adenoviruses according to the invention can be prepared by any technique known to the person skilled in the art (Levrero et al., Gene, 1991, 101:195; European Publication No. EP 185 573; Graham, EMBO J., 1984, 3:2917; Graham et al., J. Gen. Virol., 1977, 36:59). Recombinant adenoviruses are recovered and purified using standard molecular biological techniques that are well known to one of ordinary skill in the art.

Adeno-associated viruses. The adeno-associated viruses (AAV) are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see, PCT Publication Nos. WO 91/18088 and WO 93/09239; U.S. Pat. Nos. 4,797,368 and 5,139,941; European Publication No. EP 488 528). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

Retrovirus vectors. In another embodiment the gene can be introduced in a retroviral vector, e.g., as described in U.S. Pat. No. 5,399,346; Mann et al., Cell 1983, 33:153; U.S. Pat. Nos. 4,650,764 and 4,980,289; Markowitz et al., J. Virol. 1988, 62:1120; U.S. Pat. No. 5,124,263; European Publication Nos. EP 453 242 and EP178 220; Bernstein et al., Genet. Eng.1985, 7:235; McCormick, BioTechnology 1985, 3:689; PCT Publication No. WO 95/07358; and Kuo et al., Blood 1993, 82:845. The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukemia virus”) MSV (“murine Moloney sarcoma virus”), HaSV (“Harvey sarcoma virus”); SNV (“spleen necrosis virus”); RSV (“Rous sarcoma virus”) and Friend virus. Suitable packaging cell lines have been described in the prior art, in particular the cell line PA317 (U.S. Pat. No. 4,861,719); the PsiCRIP cell line (PCT Publication No. WO 90/02806) and the GP+envAm-12 cell line (PCT Publication No. WO 89/07150). In addition, the recombinant retroviral vectors can contain modifications within the LTRs for suppressing transcriptional activity as well as extensive encapsidation sequences that may include a part of the gag gene (Bender et al., J. Virol. 1987, 61:1639). Recombinant retroviral vectors are purified by standard techniques known to those having ordinary skill in the art.

Retroviral vectors can be constructed to function as infectious particles or to undergo a single round of transfection. In the former case, the virus is modified to retain all of its genes except for those responsible for oncogenic transformation properties, and to express the heterologous gene. Non-infectious viral vectors are manipulated to destroy the viral packaging signal, but retain the structural genes required to package the co-introduced virus engineered to contain the heterologous gene and the packaging signals. Thus, the viral particles that are produced are not capable of producing additional virus.

Retroviral vectors can also be introduced by DNA viruses that permit one cycle of retroviral replication and amplifies transfection efficiency (see PCT Publication Nos. WO 95/22617, WO 95/26411, WO 96/39036 and WO 97/19182).

Lentivirus vectors. In another embodiment, lentiviral vectors can be used as agents for the direct delivery and sustained expression of a transgene in several tissue types, including brain, retina, muscle, liver and blood. The vectors can efficiently transduce dividing and nondividing cells in these tissues, and maintain long-term expression of the gene of interest. For a review, see, Naldini, Curr. Opin. Biotechnol. 1998, 9:457-63; see also Zufferey, et al., J. Virol. 1998, 72:9873-80). Lentiviral packaging cell lines are available and known generally in the art. They facilitate the production of high-titer lentiviral vectors for gene therapy. An example is a tetracycline-inducible VSV-G pseudotyped lentivirus packaging cell line that can generate virus particles at titers greater than 10⁶ IU/ml for at least 3 to 4 days (Kafri, et al., J. Virol. 1999, 73: 576-584). The vector produced by the inducible cell line can be concentrated as needed for efficiently transducing non-dividing cells in vitro and in vivo.

Non-viral vectors. In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner, et. al., Proc. Natl. Acad. Sci. USA 1987, 84:7413-7417; Felgner and Ringold, Science 1989, 337:387-388; see Mackey, et al., Proc. Natl. Acad. Sci. USA 1988, 85:8027-8031; Ulmer et al., Science 1993, 259:1745-1748). Useful lipid compounds and compositions for transfer of nucleic acids are described in PCT Patent Publication Nos. WO 95/18863 and WO 96/17823, and in U.S. Pat. No. 5,459,127. Lipids may be chemically coupled to other molecules for the purpose of targeting (see Mackey, et. al., supra). Targeting peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., PCT Patent Publication No. WO 95/21931), peptides derived from DNA binding proteins (e.g., PCT Patent Publication No. WO 96/25508), or a cationic polymer (e.g., PCT Patent Publication No. WO 95/21931).

It is also possible to introduce the vector in vivo as a naked DNA plasmid. Naked DNA vectors for gene therapy can be introduced into the desired host cells by methods known in the art, e.g., electroporation, microinjection, cell fusion, DEAE dextran, calcium phosphate precipitation, use of a gene gun, or use of a DNA vector transporter (see, e.g., Wu et al., J. Biol. Chem. 1992, 267:963-967; Wu and Wu, J. Biol. Chem. 1988, 263:14621-14624; Canadian Patent Application No. 2,012,311; Williams et al., Proc. Natl. Acad. Sci. USA 1991, 88:2726-2730). Receptor-mediated DNA delivery approaches can also be used (Curiel et al., Hum. Gene Ther. 1992, 3:147-154; Wu and Wu, J. Biol. Chem. 1987, 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci. 1988, 321:893; PCT Publication Nos. WO 99/01157, WO 99/01158, and WO 99/01175).

### Therapeutic Use of Constructs

The invention also provides methods for treating adenocarcinomas by administration of a therapeutic of the invention. Such therapeutics include the constructs of the invention and nucleic acids encoding the constructs of the invention.

Generally, administration of products of a species origin or species reactivity that is the same species as that of the subject is preferred. Thus, in administration to humans, the therapeutic methods of the invention preferably use a construct that is derived from a human immunoglobulin superfamily protein but may be an immunoglobulin superfamily protein from a heterologous species such as, for example, a mouse that may or may not be humanized; in other embodiments, the methods of the invention use a modified antibody that is derived from a chimeric or humanized antibody.

Specifically, therapeutic compositions containing the constructs of the invention that specifically bind a particular molecule can be used in the treatment or prevention of diseases or disorders associated with the expression of the particular molecule, e.g., mucin.

The subjects to which the present invention is applicable may be any mammalian or vertebrate species that include, but are not limited to, cows, horses, sheep, pigs, fowl (e.g., chickens), goats, cats, dogs, hamsters, mice, rats, monkeys, rabbits, chimpanzees, and humans. In a preferred embodiment, the subject is a human.

### Treatment and Prevention of Cancers

The invention provides methods of treating or preventing cancers. The method includes administering to a subject in need of such treatment or prevention a therapeutic of the invention, i.e., a construct, that may or may not bind to mucin.

Cancers, including, but not limited to, neoplasms, tumors, metastases, or any disease or disorder characterized by uncontrolled cell growth, in which the tumor cells express a mucin, particularly MUC-1, can be treated or prevented by administration of a construct of the invention. Whether a particular therapeutic is effective to treat or prevent a certain type of cancer can be determined by any method known in the art, for example but not limited to, the methods described in infra.

In other embodiments of the invention, the subject being treated with the construct may, optionally, be treated with other cancer treatments such as surgery, radiation therapy, or chemotherapy. In particular, the therapeutic of the invention used to treat or prevent cancer may be administered in conjunction with one or a combination of chemotherapeutic agents including, but not limited to, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, docetaxel, etc.

### Gene Vaccines

In a specific embodiment, vectors comprising a sequence encoding a construct of the invention are administered to treat or prevent a disease or disorder associated with the expression or function of a molecule.

Any of the methods for gene therapy available in the art can be used according to the present invention. Exemplary methods are described below.

For general reviews of the methods of gene therapy, see, Goldspiel et al., Clinical Pharmacy 1993, 12:488-505; Wu and Wu, Biotherapy 1991, 3:87-95; Tolstoshev, Ann. Rev. Pharmacol. Toxicol. 1993, 32:573-596; Mulligan, Science 1993, 260:926-932; and Morgan and Anderson, Ann. Rev. Biochem. 1993, 62:191-217; and May, TIBTECH 1993, 11:155-215. Methods commonly known in the art of recombinant DNA technology that can be used are described in Ausubel et al., (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al., (eds.), 1994, Current Protocols in Human Genetics, John Wiley & Sons, NY. Vectors suitable for gene therapy are described above.

In one aspect, the therapeutic vector comprises a nucleic acid that expresses the construct in a suitable host. In particular, such a vector has a promoter operationally linked to the coding sequence for the construct. The promoter can be inducible or constitutive and, optionally, tissue-specific. In another embodiment, a nucleic acid molecule is used in which the antibody coding sequences and any other desired sequences are flanked by regions that promote homologous recombination at a desired site in the genome, thus providing for expression of the construct from a nucleic acid molecule that has integrated into the genome (Koller and Smithies, Proc. Natl. Acad. Sci. USA 1989, 86:8932-8935; Zijlstra et al., Nature 1989, 342:435-438).

Delivery of the vector into a patient may be either direct, in which case the patient is directly exposed to the vector or a delivery complex, or indirect, in which case, cells are first transformed with the vector in vitro then transplanted into the patient. These two approaches are known, respectively, as in vivo and ex vivo gene therapy.

In a specific embodiment, the vector is directly administered in vivo, where it enters the cells of the organism and mediates expression of the constructs. This can be accomplished by any of numerous methods known in the art, e.g., by constructing it as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont); or coating with lipids or cell-surface receptors or transfecting agents, encapsulation in biopolymers (e.g., poly-β1-4-N-acetylglucosamine polysaccharide; see, U.S. Pat. No. 5,635,493), encapsulation in liposomes, microparticles, or microcapsules; by administering it in linkage to a peptide or other ligand known to enter the nucleus; or by administering it in linkage to a ligand subject to receptor-mediated endocytosis (see, e.g., Wu and Wu, J. Biol. Chem. 1987,62:4429-4432), etc. In another embodiment, a nucleic acid ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188). These methods are in addition to those discussed above in conjunction with “Viral and Non-viral Vectors”.

Alternatively, single chain antibody-like constructs can also be administered, for example, by expressing nucleotide sequences encoding single-chain antibodies within the target cell population by utilizing, for example, techniques such as those described in Marasco et al. (Proc. Natl. Acad Sci. USA 1993, 90:7889-7893).

The form and amount of therapeutic nucleic acid envisioned for use depends on the type of disease and the severity of the desired effect, patient state, etc., and can be determined by one skilled in the art.

### Vaccine Formulations and Administration

The invention also provides vaccine formulations containing therapeutics of the invention, which vaccine formulations are suitable for administration to elicit a protective immune (humoral and/or cell mediated) response against cancer cells bearing mucins, e.g., for the treatment and prevention of diseases.

Suitable preparations of such vaccines include injectables, either as liquid solutions or suspensions; solid forms suitable for solution in, suspension in, liquid prior to injection, may also be prepared. The preparation may also be emulsified, or the constructs antibodies encapsulated in liposomes. The active immunogenic ingredients are often mixed with excipients that are pharmaceutically acceptable and compatible with the active ingredient. Suitable excipients are, for example, water, saline, buffered saline, dextrose, glycerol, ethanol, sterile isotonic aqueous buffer or the like and combinations thereof. In addition, if desired, the vaccine preparation may also include minor amounts of auxiliary substances such as wetting or emulsifying agents, pH buffering agents, and/or adjuvants that enhance the effectiveness of the vaccine. The construct when prepared as a vaccine can be introduced in microspheres or microcapsules, e.g., prepared from PGLA (see, U.S. Pat. Nos. 5,814,344, 5,100,669, and 4,849,222; PCT Publication Nos. WO 95/11010 and WO 93/07861).

The effectiveness of an adjuvant may be determined by measuring the induction of anti-idiotype antibodies directed against the injected construct formulated with the particular adjuvant.

The composition can be a liquid solution, suspension, emulsion, tablet, pill, capsule, sustained release formulation, or powder. For oral administration, the therapeutics can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups, emulsions or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives Or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Generally, the ingredients are supplied either separately or mixed together in unit dosage form, for example, as a dry lyophilized powder or water-free concentrate in a sealed container such as a vial or sachette indicating the quantity of active agent. Where the composition is administered by injection, an ampoule of sterile diluent can be provided so that the ingredients may be mixed prior to administration.

In a specific embodiment, the lyophilized construct of the invention is provided in a first container; a second container comprises diluent consisting of an aqueous solution of 50% glycerin, 0.25% phenol, and an antiseptic (e.g., 0.005% brilliant green).

The invention also provides a pharmaceutical pack or kit comprising one or more containers filled with one or more of the ingredients of the vaccine formulations of the invention. Associated with such container(s) can be a notice in the form prescribed by a governmental agency regulating the manufacture, use or sale of pharmaceuticals or biological products, which notice reflects approval by the agency of manufacture, use or sale for human administration.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. Composition comprising a compound of the invention formulated in a compatible pharmaceutical carrier may also be prepared, placed in an appropriate container, and labeled for treatment of an indicated condition.

Many methods may be used to introduce the vaccine formulations of the invention; these include but are not limited to oral, intracerebral, intradermal, intramuscular, intraperitoneal, intravenous, subcutaneous, intranasal routes, and via scarification (scratching through the top layers of skin, e.g., using a bifurcated needle) or any other standard routes of immunization.

### Effective Dose

The constructs and vectors described herein can be administered to a patient at therapeutically effective doses to treat mucin-bearing adenocarcinomas. A therapeutically effective dose refers to that amount of a therapeutic sufficient to result in a healthful benefit in the treated subject.

The precise dose of the constructs to be employed in the formulation depends on the route of administration, and the nature of the patient's cancer, and should be decided according to the judgment of the practitioner and each patient's circumstances according to standard clinical techniques. An effective dose is an amount effective to result in development of an effective anti-tumor immune response in vivo. The ability of a therapeutic composition of the invention to produce this effect can be detected in vitro, e.g., using a binding assay with labeled construct as exemplified infra. Such an assay can be formatted in a solid phase format, in which a mucin is adsorbed to a solid support, or in a cell-based assay format. Effective doses may be extrapolated from dose-response curves derived from animal model test systems, including transgenic animal models.

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutics that exhibit large therapeutic indices are preferred. While therapeutics that exhibit toxic side effects can be used, care should be taken to design a delivery system that targets such compounds to the site of affected tissue in order to minimize potential damage to unaffected cells and, thereby, reduce side effects.

The data obtained from the cell culture assays and animal studies can be used in formulating a range of dosage for use in humans. The dosage lies preferably within a range of circulating concentrations that include the ED₅₀ with little or no toxicity. The dosage can vary within this range depending upon the dosage form employed and the route of administration utilized. For any construct used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

### EXAMPLES

The following Examples illustrate the invention without limiting it.

### Example 1

### Construction of Variable Region Gene Containing the CDR Sequences from HMFG-1

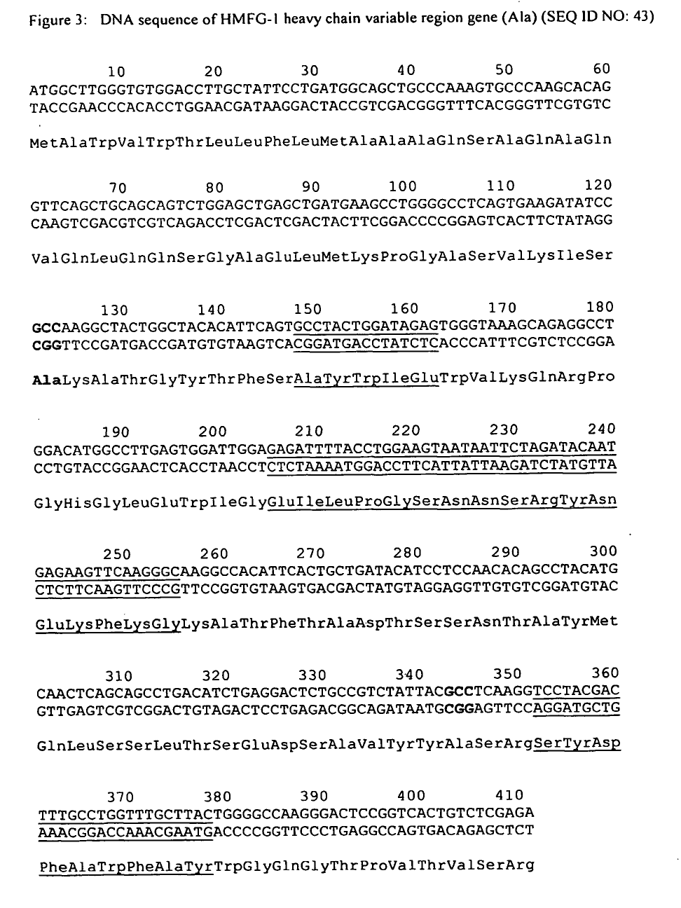

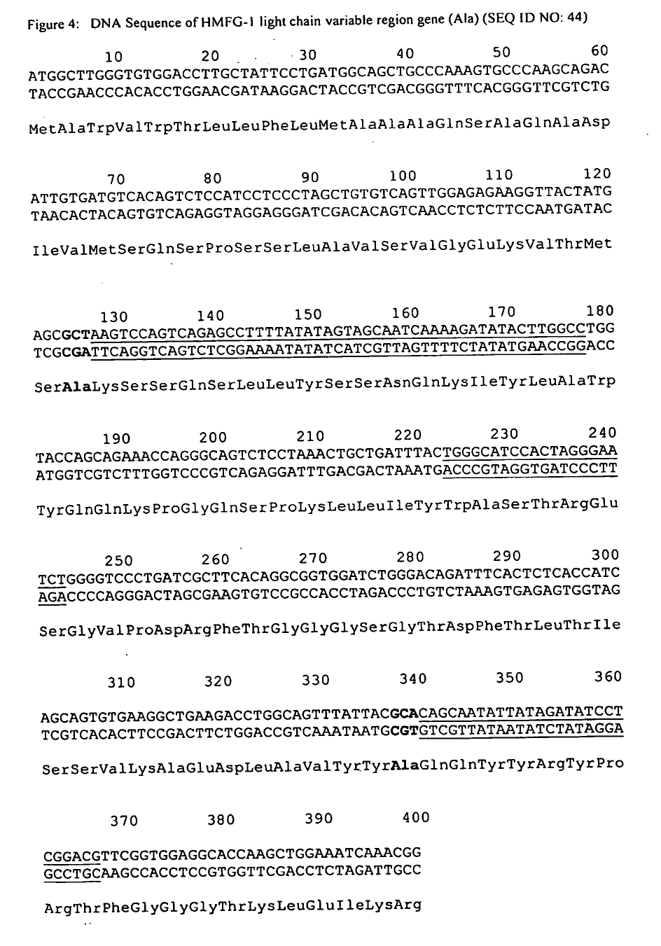

Heavy and light chain variable region genes were constructed containing framework and CDR sequences from the monoclonal antibody HMFG-1 (FIGS. 3 and 4). The engineered genes were made by assembling overlapping oligonucleotides that were from 65 to 72 nucleotides in length using standard conditions (Table 1). A second set of heavy and light chain variable region genes were also constructed in which specific cysteine residues, known to form intra-chain disulfide bonds, were changed to alanine residues. Cysteine residues at positions 22 and 96 of the heavy chain and 23 and 88 of the light chain were changed to alanine residues. The assembled variable region genes were joined to appropriate constant region genes and then inserted into an expression vector as described below.

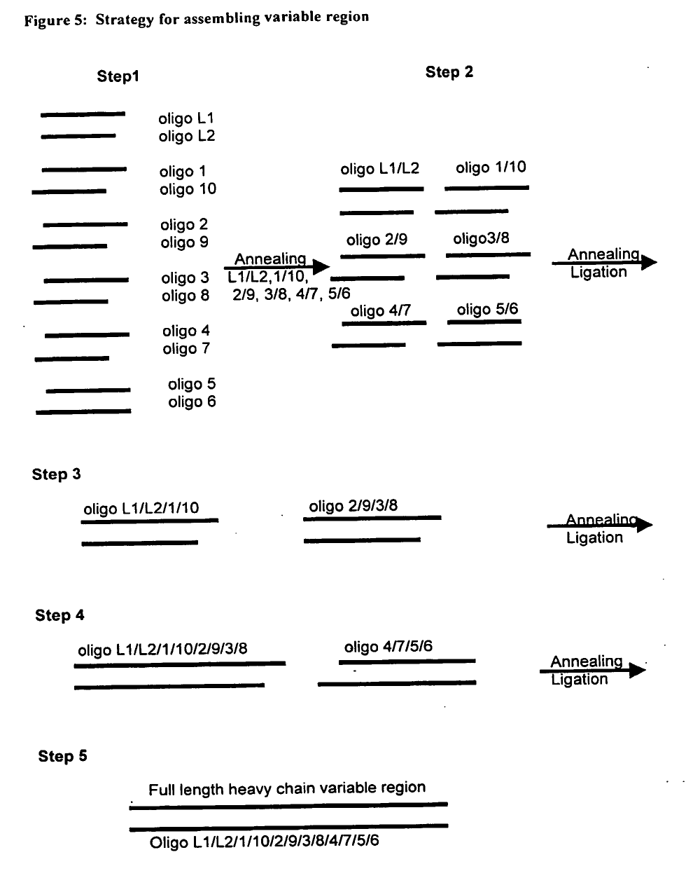

To construct the variable region genes encoding the HMFG-1 CDR sequences and lacking the intra-chain disulfide bonds, the following steps were performed (FIG. 5). Purified, single stranded oligodeoxynucleotides were annealed together to create cohesive, double stranded DNA fragments. The specific sequences of the oligonucleotides used to construct heavy and light chain variable region genes containing the CDR sequences from HMFG are presented in Table 1 and Table 2, respectively. The double stranded DNA fragments have cohesive, single stranded ends of six to nine bases that are required for joining the individual fragments in the following steps. In the next step, two annealed, double stranded DNA fragments are ligated together using standard conditions and this process is continued until the assembly of the full-length variable region gene is completed. The mixture of ligated fragments is then separated on an agarose gel and the full-length fragment is isolated and purified using a QIAEX II gel extraction kit according to the manufacturer's instructions.

To change the alanine residues present in the variable region genes back to cysteine residues, oligodeoxynucleotides were prepared (Table 3) and used with a QuikChange site-directed mutagenesis kit from Stratagene according to the manufacturer's instructions. The amino acid changes were confirmed by DNA sequencing using an ABI 310 Genetic Analyzer.

TABLE 1

Oligodeoxynucleotides used to assemble HMPG-1
heavy chain variable region gene (Ala). Sequences
are shown in 5′ to 3′ orientation and those
positions in the oligonucleotides that have been
used to convert C to A are indicated in bold and
underlined.

| | |
|---|---|
| Leader sequence L1 | |
| GAATTCATGGCTTGGGTGTGGACCTTGCTATTCCTGATGGCAGCTGCCCA | (SEQ ID NO: 9) |
| AAGTGCCCAAGCA | |
| Leader sequence L2 | |
| TGAAACCCGTCGACGGTAGTCCTTATCGTTCCAGGTGTGGGTTCGGTAGA | (SEQ ID NO: 10) |
| ATTC | |
| HMFG VHA1 | |
| CAGGTTCAGCTGCAGCAGTCTGGAGCTGAGCTGATGAAGCCTGGGGCCTC | (SEQ ID NO: 11) |
| AGTGAAGATATCCGCCAAGGCTACT′ | |
| HMFG VHA2 | |
| GGCTACACATTCAGTGCCTACTGGATAGAGTGGGTAAAGCAGAGGCCTGG | (SEQ ID NO: 12) |
| ACATGGCCTTGAGTGGATT | |
| HMFG VHA3 | |
| GGAGAGATTTTACCTGGAAGTAATAATTCTAGATACAATGAGAAGTTCAA | (SEQ ID NO: 13) |
| GGGCAAGGCCACATTCACTGCTGAT | |
| HMFG VHA4 | |
| ACATCCTCCAACACAGCCTACATGCAACTCAGCAGCCTGACATCTGAGGA | (SEQ ID NO: 14) |
| CTCTGCCGTCTATTAC | |
| HMFG VHA5 | |
| GCCTCAAGGTCCTACGACTTTGCCTGGTTIGCTTACTGGGGCCAAGGGAC | (SEQ ID NO: 15) |
| TCCGGTCACTGTCTCTGCAGAATTC | |
| HMFG VHA6 | |
| GAATTCTGCAGAGACAGTGACCGGAGTCCCTTGGCCCCAGTAAGCAAACC | (SEQ ID NO: 16) |
| AGGCAAAGTCGTAGGACCTTGAGGCGTAATAGAC | |
| HMFG VHA7 | |
| GGCAGAGTCCTCAGATGTCAGGCTGCTGAGTTGCATGTAGGCTGTGTTGG | (SEQ ID NO: 17) |
| AGGATGTATCAGCAGT | |
| HMFG VHA8 | |
| AATGTGGCCTTGCCCTTGAACTTCTCATTGTATCTAGAATTATTACTTCC | (SEQ ID NO: 18) |
| AGGTAAAATCTCTCCAATCCACTC | |
| HMFG VHA9 | |
| AAGGCCATGTCCAGGCCTCTGCTTTACCCACTCTATCCAGTAGGCACTGA | (SEQ ID NO: 19) |
| ATGTGTAGCCAGTAGCCTT | |
| HMPG VHA10 | |
| GGCGGATATCTTCACTGAGGCCCCAGGCTTCATCAGCTCAGCTCCAGACT | (SEQ ID NO: 20) |
| GCTGCAGCTGAACCTGTGCTTGGGC | |

TABLE 2

Oligodeoxyribonucleotides used to assemble HMFG-1
light chain variable region gene (Ala). Sequences
are shown in 5′ to 3′ orientation and those
positions in the oligonucleotides that have been
used to convert C to A are indicated in bold and
underlined.

| | |
|---|---|
| HMPG VLA | |
| GACATTGTGATGTCACAGTCTCCATCCTCCCTAGCTGTGTCAGTTGGAGA | (SEQ ID NO: 21) |
| GAAGGTTACTATGAGCGCTAAGTCCAGT | |
| HMPG VLA2 | |
| CAGAGCCTTTTATATAGTAGCAATCAAAAGATCTACTTGGCCTGGTACCA | (SEQ ID NO: 22) |
| GCAGAAACCAGGGCAGTCTCCTAAA | |
| HMFG VLA3 | |
| CTGCTGATTTACTGGCATCCACTAGGGAATCTGGGGTCCCTGATCGCTTC | (SEQ ID NO: 23) |
| ACAGGCGGTGGATCTGGG | |
| HMFG VLA4 | |
| ACAGATTTCACTCTCACCATCAGCAGTGTGAAGGCTGAAGACCTGGCAGT | (SEQ ID NO: 24) |
| TTATTAC | |
| HMFG VLA5 | |
| GCACAGCAATATTATAGATATCCTCGGACGTTCGGTGGAGGCACCAAGCT | (SEQ ID NO: 25) |
| GGAAATCAAACGGGAATTC | |
| HMFG VLA6 | |
| GAATTCCCGTTTGATTTCCAGCTTGGTGCCTCCACCGAACGTCCGAGGAT | (SEQ ID NO: 26) |
| ATCTATAATATTGCTGTGCGTAATAAAC | |
| HMFG VLA7 | |
| ACGGTCCAGAAGAGCCTTCACACTGCTGATGGTGAGAGTGAAATCTGTCC | (SEQ ID NO: 27) |
| CAGATCC | |
| HMFG VLA8 | |
| ACCGCCTGTGAAGCGATCAGGGACCCCAGATTCCCTAGTGGATGCCCAGT | (SEQ ID NO: 28) |
| AAATCAGCAGTTTAGGAGA | |
| HMFG VLA9 | |
| CTGCCTGGTTTCTGCTGGTACCAGGCCAAGTAGATCTTTTGATTGCTACT | (SEQ ID NO: 29) |
| ATATAAAAGGCTCTGACTGGACTTAGCGCT | |
| HMFG VLA10 | |
| CATAGTAACCTTCTCTCCAACTGACACAGCTAGGGAGGATGGAGACTGTG | (SEQ ID NO: 30) |
| ACATCACAATGTCTGCTTGGGC | |

TABLE 3

Sequences of oligonucleotides used for site
directed mutagenesis to switch Ala to Cys in heavy
and light chain variable region. Sequences are
shown in 5′ to 3′ orientation.

HMFG VL 115 1 Ala->CysGGCAGTTTATTACTGCC
AGCAATATTATAGATATCCTCGG (SEQ ID NO: 31)
HMFG VL 115 2 Ala->CysCCAGGATATCTATAATAT
TGCTGGCAGTAATAAACTGCC (SEQ ID NO: 32)
HMFG VL 44 1 Ala->CysGGTTACTATGAGCTGCAA
GTCCAGTCGAGC (SEQ ID NO: 33)
HMFG VL 44 2 Ala->CysGCTCTGACTGGACTTGCA
GCTCATAGTAACC (SEQ ID NO: 34)
HMFG HV 117 1 Ala->CysGCCGTCTATAACTGCTCA
AGGTCCTACGAC (SEQ ID NO: 35)
HMFG HV 117 2 Ala->CysGTCGTAGGACCTTGAGCA
GTAATAGACGGC (SEQ ID NO: 36)
HMFG HV 43 1 Ala->CysGTGAAGATATCCTGCAAG
GCTACTGGCTAC (SEQ ID NO: 37)
HMFG VL 43 2 Ala->CysGTAGCCAGTAGCCTTGCA
GGATATCTTCA (SEQ ID NO: 38)

The assembled, modified variable region genes containing the HMFG-1 sequences were then linked to the appropriate constant region clones. For assembly of the heavy chain of the antibody, a unique XhoI restriction enzyme site was engineered into both the 3′end of the variable region and the 5′ end of the IgG₁ heavy chain constant region. At the 5′end of the variable region, an EcoRI restriction site and a Kozak sequence were added using polymerase chain reactions (PCR). The modified heavy chain variable region was then joined to the heavy chain constant region by inserting the EcoRI/XhoI cut variable region fragment into a vector containing the EcoRI/XhoI cut heavy chain constant region

For assembly of the light chain of the antibody, a unique BglII restriction enzyme site was engineered into the 3′end of the light chain variable region and a BclI restriction enzyme site was added to the 5′end of the light chain constant region (κ chain). Similar to the heavy chain variable region, an EcoRI restriction site and Kozak sequence were added to the 5′end of the light chain variable region using PCR. When BglII and BclI cut their respective cleavage sites, both enzymes leave overhangs with the same DNA sequence that allows them to be ligated. Consequently, the modified light chain variable region clone was digested with EcoRI/BglII and the resulting fragment inserted into a vector containing the EcoRI/BclI cut light chain constant region.

In a final step, the heavy chain expression vector, containing the heavy chain variable region, and the light chain expression vector, containing the light chain variable region, were assembled into a single “double gene” expression vector. To assemble the “double gene” vector, the heavy chain expression vector is cleaved with BamHI and NotI. The resulting fragment contains the complete heavy chain expression cassette including the CMV promoter, the assembled heavy chain and a transcriptional terminator. The light chain expression vector was also cleaved with BamHI and NotI and after purifying the vector from a small fragment, the heavy chain expression cassette was inserted into the light chain vector.

Peptide sequence of the HMFG-1 heavy chain variable region. The HMFG-1 CDR sequences are underlined. Alanine residues that replaced cysteine residues are shown in bold. MetAlaTrpValTrpThrLeuLeuPheLeuMetAlaAlaAlaGlnSerAlaGlnAlaGlnValGlnLeuGlnGlnSerGlyAlaGluLeuMetLysProGlyAlaSerValLysleSerAlaLysAlaThrGlyTyrThrPheSerAlaTyrTrpIleGluTrpValLysGlnArgProGlyHisGlyLeuGluTrpIleGlyGluIleLeuProGlySerAsnAsnSerArgTyrAsnGluLysPheLysGlyLysAlaThrPheThrAlaAspThrSerSerAsnThrAlaTyrMetGlnLeuSerSerLeuThrSerGluAspSerAlaValTyrTyrAlaSerArgSerTyrAspPheAlaTrpPheAlaTyrTrpGlyGlnGlyThrProValThrValSerArg (SEQ ID NO: 39)

The nucleotide sequence of the HMFG-1 heavy chain variable region sequence is also shown. The HMFG-1 CDR sequences are underlined. Alanine codons that replaced cysteine codons are shown in bold.

(SEQ ID NO: 40)

ATGGCTTGGGTGTGGACCTTGCTATTCCTGATGGCAGCTGCCCAAAGTGC
CCAAGCACAGGTTCAGCTGCAGCAGTCTGGAGCTGAGCTGATGAAGCCTG
GGGCCTCAGTGAAGATATCCGCCAAGGCTACTGGCTACACATTCAGTGCC
TACTGGATAGAGTGGGTAAAGCAGAGGCTGGACATGGCCCTTGAGTGGAT
TGGAGAGATTTTACCTGGAAGTAATAATTCTAGATACAATGAGAAGTTCA
AGGGCAAGGCCACATTCACTGCTGATACATCCTCCAACACAGCCTACATG
CAACTCAGCAGCCTGACATCTGAGGACTCTGCCGTCTATTACGCCTCAAG
GTCCTACGACTTTGCCTGGTTTGCTTACTGGGGCCAAGGGACTCGGGTCA
CTGTCTCGAGA

Peptide sequence of the HMFG-1 light chain variable region. The HMFG-1 CDR sequences are underlined. Alanine residues that replaced cysteine residues are shown in bold.

MetAlaTrpValTrpThrLeuLeuPheLeuMetAlaAlaAlaGlnSerAlaGlnAlaAspIeValMetSerGlnSerProSerSerLeuAlaValSerValGlyGluLysValThrMetSerAlaLysSerSerGlnSerLeuLeuTyrSerSerAsnGlnLysIleTyrLeuAlaTrpTyrGlnGlnLysProGlyGlnSerProLysLeuLeuIleTyrTrpAlaSerThrArgGluSerGlyValProAspArgPheThrGlyGlyGlySerGlyThrAspPheThrLeuThrIleSerSerValLysAlaGluAspLeuAlaValTyrTyrAlaGlnGlnTurTyrArgTyrProArgThrPheGlyGlyGlyThrLysLeuGlulleLysArg (SEQ ID NO: 41)

The nucleotide sequence of the HMFG-1 light chain variable region is also shown. The HMFG-1 CDR sequences are underlined. Alanine codons that replaced cysteine codons are shown in bold.

(SEQ ID NO: 42)

ATGGCYGGGTGTGGACCTTGCTATTCCTGATGGCAGCTGCCCCAAAGTGC
CCAAGCAGACATTGTGATGTCACAGTCTCCATCCTCCCTAGCTGTGTCAG
TTGGAGAGAAGGTTACTATGAGCGCTAAGTCCAGTCAGAGCCTTTTATAT
AGTAGCAATCAAAAGATATACTTGGCCTGGTACCAGCAGAAACCAGGGCA
GTCTCCTAAACTGCTGATTTACTGGGCATCCACTAGGGAATCTGGGGTCC
CTGATCGCTTCACAGGCGGTGGATCTGGGACAGATTTCACTCTCACCATC
AGCAGTGTGAAGGCTGAAGACCTGGCAGTTTATTACGCACAGCAATATTA
TAGATATCCTCGGACGTTCGGTGGAGGCACCAAGCTGGAAATCAAACGG

### Example 2

### Protein Expression

Once constructs were prepared, initial transfections were performed transiently in CHO-K1 cells. Cotransfections were performed using two single gene constructs and a cationic liposomal reagent. Expression was measured at day 3 and day 7 by ELISA assay. The expressed CD28 synthebody was purified using Protein-A or Protein-G column chromatography and characterized by HPLC and Western immunoblotting.

Stable transfectants can be produced in a number of cell lines including but not limited to CHO-K1, NSO and HEK-293. The choice of which cell line to use will rely on a number of factors, some of which include: glycosylation patterns, expression level and ability to adapt to serum-free or protein-free media.

### Example 3

### Binding of HMFG-1 Synthebody/Vaccines of OVCAR Cells

Experimental Procedures

Flow cytometry for evaluation of binding of HMFG-1 synthebody/vaccine to OVCAR-3 cells. OVCAR-3 (purchased from ATCC) cells were distributed into 1.5-ml Eppendorf tubes at 1×10⁶ cell each and centrifuged using an Eppendorf microcentrifuge at room temperature at 6000 RPM for 1 min. The supernatant was removed by vacuuming and cells were resuspended in 1% BSA-PBS (FACS buffer) or culture supernatants containing HMFG-1 synthetic antibodies or control antibodies. Following incubation at 4° C. for 30-40 min., cells were washed once with cold FACS buffer, 0.5 mil/tube by centrifugation at 6000 RPM for 1 min and resuspended in 50 ml of FACS buffer. Two microliters of FITC-labeled goat-anti-human IgG or goat-anti-mouse IgG₁ were added to each tube. The cells were incubated at 4° C. for 30 min. and then washed twice with cold FACS buffer. Finally, cells were resuspended in 0.4 ml of FACS buffer, acquired on a flow cytometer and analyzed using software.

Results

OVCAR cells were incubated with chimeric (A) or murine (B) HMFG-1 synthetic antibody/vaccines and control antibodies (IgG₁ and consensus antibody). The binding of the HMFG-1 synthetic antibody/vaccines and control antibodies to the cells was probed with FITC-labeled goat-anti-human or mouse IgG and evaluated by flow cytometry. The results are expressed as mean of percentage of positive cells±S.D. (FIG. 3). As indicated in FIGS. 3A and 3B, several versions of the vaccine were tested. C/A version: The cysteine involved in the formation of intra-chain disulfide bond in the CDR region of the light chain was replaced by alanine. A/C version: The cysteine involved in the formation of intra-chain disulfide bond in the CDR region of the heavy chain was replaced by alanine. C/C version: No cysteine replacement was performed.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, applications, publications, test methods, literature, and other materials cited herein are hereby incorporated by reference.

What is claimed is:

1. A variant of an immunoglobulin variable domain, said immunoglobulin variable domain comprising (A) at least one CDR region and (B) framework regions flanking said CDR, said variant comprising:
(a) said CDR region having added or substituted therein at least one binding sequence and
(b) said flanking framework regions,
wherein said binding sequence is heterologous to said CDR and is an antigenic sequence from a MUC-1 binding sequence.

2. The variant as define in claim 1, wherein the variable domain lacks an intrachain disulfide bond.

3. A variant as defined in claim 1, wherein (i) one or more amino acid residues in one or more of said flanking framework regions has been substituted or deleted, (ii) one or more amino acid residues has been added in one or more of said flanking framework regions, or (iii) a combination of (i) and (ii).

4. A variant as defined in claim 1, wherein (i) one or more amino acid residues in one or more framework regions other than said framework regions flanking said CDR has been substituted or deleted, (ii) one or more amino acid residues has been added in one or more framework regions other than said framework regions flanking said CDR, or (iii) a combination of (i) and (ii).

5. A variant as defined in claim 1, wherein (i) one or more amino acid residues in one or more of said flanking framework regions has been substituted or deleted, (ii) one or more amino acid residues has been added in one or more of said flanking framework regions, or (iii) a combination of (i) and (ii); and wherein (iv) one or more amino acid residues in one or more framework regions other than said framework regions flanking said CDR has been substituted or deleted, (v) one or more amino acid residues has been added in one or more framework regions other than said framework regions flanking said CDR, or (vi) a combination of (iv) and (v).

6. A variant of an immunoglobulin variable domain, said immunoglobulin variable domain comprising (A) at least one CDR region and (B) framework regions flanking said CDR, said variant comprising:
(a) said CDR region having added or substituted therein at least one amino acid sequence which is heterologous to said CDR and
(b) said flanking framework regions,
wherein said heterologous sequence is an antigenic sequence from a thrombopoietin receptor binding sequence.

7. A variant as defined in claim 6, wherein the variable domain lacks an intrachain disulfide bond.

8. A variant as defined in claim 6, wherein (i) one or more amino acid residues in one or more of said flanking framework regions has been substituted or deleted, (ii) one or more amino acid residues has been added in on or more of said flanking framework regions, or (iii) a combination of (i) and (ii).

9. A variant as defined in claim 6, wherein (i) one or more amino acid residues in one or more framework regions other than said framework regions flanking said CDR has been substituted or deleted, (ii) one or more amino acid residues has been added in one or more framework regions other than said framework regions flanking said CDR, or (iii) a combination of (i) and (ii).

10. A variant as defined in claim 6, wherein (i) one or more amino acid residues in one or more of said flanking framework regions has been substituted or deleted, (ii) one or more amino acid residues has been added in one or more of said flanking framework regions, (iii) a combination of (i) and (ii); and wherein (iv) one or more amino acid residues in one or more framework regions other than said framework regions flanking said CDR has been substituted or deleted, (v) one or more amino acid residues has been added in one or more framework regions other than said framework regions flanking said CDR, or (vi) a combination of (iv) and (v).

11. A variant as defined in claim 6, wherein said CDR is more than one CDR.

12. A variant as defined in claim 6, wherein said heterologous sequence is a CDR of a heavy chain variable region.

13. A variant as defined in claim 6, wherein said heterologous sequence is a CDR of a light chain variable region.

14. A variant as defined in claim 6, wherein said antigenic sequence is selected from the group consisting of SEQ ID Nos: 1-6.

15. A variant as defined in claim 6, which is an antibody.

16. A molecule comprising a variant as defined in claim 6.

17. A molecule comprising a variant as defined in claim 7.

18. A molecule comprising a variant as defined in claim 8.

19. A molecule comprising a variant as defined in claim 9.

20. A molecule comprising a variant as defined in claim 10.

21. A molecule comprising a variant as defined in claim 14.

22. A molecule as defined in claim 16, further comprising one or more constant domains from an immunoglobulin.

23. A molecule as defined in claim 16, further comprising a second variable domain linked to said variant.

24. A molecule as defined in claim 16, further comprising a second variable domain linked to said variant, and one or more constant domains from an immunoglobulin.

25. A molecule as defined in claim 16, wherein said CDR region is CDR 1.

26. A molecule as defined in claim 16, wherein said CDR region is CDR 2.

27. A molecule as defined in claim 16, wherein said CDR region is CDR 3.

28. A molecule as defined in claim 16, which is an antibody.

29. A molecule as defined in claim 16, which is derived from a human antibody.

30. A molecule as defined in claim 16, which is derived from a chimeric or a humanized antibody.

31. An immunoglobulin comprising a heavy chain and a light chain, wherein said heavy chain comprises a variant as defined in claim 6 and three constant domains from an immunoglobulin heavy chain, and said light chain comprises a second variable domain associated with said variant and a constant domain from an immunoglobulin light chain.

32. An immunoglobulin comprising a heavy chain and a light chain, wherein said light chain comprises a variant as defined in claim 6 and a constant domain from an immunoglobulin light chain, and said heavy chain comprises a second variable domain associated with said variant and three constant domains from an immunoglobulin heavy chain.

33. An isolated nucleic acid encoding a variant as defined in claim 1.

34. An isolated nucleic acid encoding a variant as defined in claim 6.

35. An isolated nucleic acid encoding a molecule as defined in claim 16.

36. An isolated nucleic acid encoding an immunoglobulin as defined in claim 29.

37. An isolated nucleic acid encoding an immunoglobulin as defined in claim 30.

38. A cell containing nucleic acid as defined in claim 31.

39. A cell containing nucleic acid as defined in claim 32.

40. A cell containing nucleic acid as defined in claim 33.

41. A cell containing nucleic acid as defined in claim 34.

42. A cell containing nucleic acid as defined in claim 35.

43. A recombinant non-human host containing nucleic acid as defined in claim 31.

44. A recombinant non-human host containing nucleic acid as defined in claim 32.

45. A recombinant non-human host containing nucleic acid as defined in claim 33.

46. A recombinant non-human host containing nucleic acid as defined in claim 34.

47. A recombinant non-human host containing nucleic acid as defined in claim 35.

48. A vaccine composition comprising a therapeutically or prophylactically effective amount of a variant as defined in claim 1, and an adjuvant.

49. A vaccine composition comprising a therapeutically or prophylactically effective amount of a variant as defined in claim 6, and an adjuvant.

50. A vaccine composition comprising a therapeutically or prophylactically effective amount of a variant as defined in claim 16, and an adjuvant.

51. A vaccine composition comprising a therapeutically or prophylactically effective amount of an immunoglobulin as defined in claim 29, and an adjuvant.

52. A vaccine composition comprising a therapeutically or prophylactically effective amount of an immunoglobulin as defined in claim 30, and an adjuvant.

53. A method of treating or preventing an epithelial cell cancer in a subject in need of such treatment or prevention, said method comprising administering to said subject a disease treating or preventing effective amount of a variant as defined in claim 1.

54. A method of treating or preventing an epithelial cell cancer in a subject in need of such treatment or prevention, said method comprising administering to said subject a disease treating or preventing effective amount of a variant as defined in claim 6.

55. A method of treating or preventing an epithelial cell cancer in a subject in need of such treatment or prevention, said method comprising administering to said subject a disease treating or preventing effective amount of a molecule as defined in claim 16.

56. A method of treating or preventing an epithelial cell cancer in a subject in need of such treatment or prevention, said method comprising administering to said subject a disease treating or preventing effective amount of an immunoglobulin as defined in claim 29.

57. A method of treating or preventing an epithelial cell cancer in a subject in need of such treatment or prevention, said method comprising administering to said subject a disease treating or preventing effective amount of a nucleic acid as defined in claim 31.

58. A method of treating or preventing an epithelial cell cancer in a subject in need of such treatment or prevention, said method comprising administering to said subject a disease treating or preventing effective amount of a vaccine composition as defined in claim 46.

59. A method of treating or preventing an epithelial cell cancer in a subject in need of such treatment or prevention, said method comprising administering to said subject a disease treating or preventing effective amount of a vaccine as defined in claim 48.

60. A variant of an immunoglobulin variable domain, said immunoglobulin variable domain comprising at least one CDR region, said variant comprising said CDR region having added or substituted therein at least one antigenic sequence from a thrombopoietin receptor binding sequence, said at least one sequence being selected from the group consisting of (a) a binding sequence heterologous to said CDR; (b) a CTL-epitope sequence; (c) a T-helper cell sequence; (d) a B-helper cell sequence; and (e) combinations thereof, wherein said at least one sequence is heterologous to said CDR and the variable domain lacks an intrachain disulfide bond.

61. A variant as claimed in claim 60 wherein said variable region comprises (a) a CDR1 region having said CTL epitope sequence substituted or added therein; (b) a CDR2 region having said T-helper cell substituted or added therein; and (c) a CDR3 region having said binding sequence of B-helper cell sequence substituted or added therein.

62. A variant as claimed in claim 60 wherein said binding sequence is selected from the group consisting of SEQ ID Nos: 1-6.

63. A variant as claimed in claim 60 which is an antibody.

64. A molecule comprising a variant as claimed in claim 60.

65. A molecule as claimed in claim 64 further comprising one or more constant domains from an immunoglobulin.

66. A molecule as claimed in claim 64 further comprising a second variable domain linked to said variant.

67. A molecule as claimed in claim 64 further comprising a second variable domain linked to said variant and one or more constant domains from an immunoglobulin.

68. A molecule as claimed in claim 64 which is an antibody.

69. A molecule as claimed in claim 64 which is derived from a human antibody.

70. A molecule as claimed in claim 64 which is derived from a chimeric or humanized antibody.

71. An immunoglobulin comprising a heavy chain and a light chain, wherein said heavy chain comprises a variant as claimed in claim 60 and three constant domains from an immunoglobulin heavy chain, and said light chain comprises a second variable domain associated with said variant and a constant domain from an immunoglobulin light chain.

72. An immunoglobulin comprising a heavy chain and a light chain, wherein said light chain comprises a variant as claimed in claim 60 and a constant domain from an immunoglobulin light chain, and said heavy chain comprises a second variable domain associated with said variant and three constant domains from an immunoglobulin heavy chain.

73. An isolated nucleic acid encoding a variant as claimed in claim 60.

74. An isolated nucleic acid encoding a molecule as claimed in claim 64.

75. An isolated nucleic acid encoding an immunoglobulin as claimed in claim 71.

76. An isolated nucleic acid encoding an immunoglobulin as claimed in claim 72.

77. A cell containing nucleic acid as claimed in claim 73.

78. A cell containing nucleic acid as claimed in claim 74.

79. A cell containing nucleic acid as claimed in claim 75.

80. A cell containing nucleic acid as claimed in claim 76

81. A recombinant non-human host containing nucleic acid as claimed in claim 74.

82. A recombinant non-human host containing nucleic acid as claimed in claim 74.

83. A recombinant non-human host containing nucleic acid as claimed in claim 75.

84. A recombinant non-human host containing nucleic acid as claimed in claim 76.

85. A vaccine composition comprising a therapeutically or prophylactically effective amount of a variant as claimed in claim 60 and an adjuvant.

86. A vaccine composition comprising a therapeutically or prophylactically effective amount of a molecule as claimed in claim 64 and an adjuvant.

87. A vaccine compostion comprising a therapeutically or prophylactically effective amount of an immunoglobulin as claimed in claim 71 and an adjuvant.

88. A vaccine compostion comprising a therapeutically or prophylactically effective amount of an immunoglobulin as claimed in claim 72 and an adjuvant.

89. A method of treating or preventing cancer in a subject in need of such treatment or prevention, said method comprising administering to said subject a disease treating or preventing effective amount of a variant as claimed in claim 60 and an adjuvant.

90. A method of treating or preventing cancer in a subject in need of such treatment or prevention, said method comprising administering to said subject a disease treating or preventing effective amount of a molecule as claimed in claim 64 and an adjuvant.

91. A method of treating or preventing cancer in a subject in need of such treatment or prevention, said method comprising administering to said subject a disease treating or preventing effective amount of an immunoglobulin as claimed in claim 71 and an adjuvant.

92. A method of treating or preventing cancer in a subject in need of such treatment or prevention, said method comprising administering to said subject a disease treating or preventing effective amount of an immunoglobulin as claimed in claim 72 and an adjuvant.

93. A method of eliciting an anti-idiotypic response to an antigen in a subject in need of treatment or prevention of a disease condition associated with said antigen, said method comprising administering to said subject a disease treating or preventing effective amount of a variant as claimed in claim 60 and an adjuvant.

94. A method of eliciting an anti-idiotypic response to an antigen in a subject in need of treatment or prevention of a disease condition associated with said antigen, said method comprising administering to said subject a disease treating or preventing effective amount of a molecule as claimed in claim 64 and an adjuvant.

95. A method of eliciting an anti-idiotypic response to an antigen in a subject in need of treatment or prevention of a disease condition associated with said antigen, said method comprising administering to said subject a disease treating or preventing effective amount of an immunoglobulin as claimed in claim 71 and an adjuvant.

96. A method of eliciting an anti-idiotypic response to an antigen in a subject in need of treatment or prevention of a disease condition associated with said antigen, said method comprising administering to said subject a disease treating or preventing effective amount of an immunoglobulin as claimed in claim 72 and an adjuvant.
